(19) United States
(12) Patent Application Publication
(10) Pub. No.: US 2023/0399379 A1
(43) Pub. Date: Dec. 14, 2023

(54) EXPRESSION OF COLLAGEN PEPTIDE COMPONENTS IN PROKARYOTIC SYSTEMS

(71) Applicants: Gelita AG, Eberbach (DE); Universität Bielefeld, Bielefeld (DE)

(72) Inventors: Severin Wedde, Bielefeld (DE); Joe Max Risse, Bielefeld (DE); Karl Friehs, Bielefeld (DE); Martin Hahn, Eberbach (DE); Benjamin Ludwig, Eberbach (DE)

(21) Appl. No.: 17/997,980

(22) PCT Filed: May 5, 2021
(86) PCT No.: PCT/EP2021/061820

§ 371 Date: Nov. 4, 2022

(30) Foreign Application Priority Data
May 6, 2020 (DE) 10 2020 205 703.6

(51) Int. Cl. C07K 14/78; C12N 15/52; C12N 15/70

(52) U.S. Cl. CPC C07K 14/78; C12N 15/70; C12N 15/52

(57) ABSTRACT

The present invention relates to methods for preparing hydroxylated collagen peptide components and to hydroxylated collagen peptide components, in particular hydroxylated collagen peptides, obtained from these methods.

### CROSS-REFERENCE TO RELATED APPLICATION

This application is a U.S. National Stage Application under 35 U.S.C. § 371 of International Application No. PCT/EP2021/061820, filed Mar. 5, 2021, which claims priority to German Patent Application No. 10 2020 205 703.6, filed on May 6, 2020. The contents of each of the which are hereby incorporated by reference in their entirety into the present disclosure.

### REFERENCE TO AN ELECTRONIC SEQUENCE LISTING

The instant application contains a Sequence Listing which has been filed electronically in ASCII format and is hereby incorporated by reference in its entirety. Said ASCII copy, updated on Nov. 10, 2022, is named 72PD-365912-US_ST25.txt and is 68,063 bytes in size.

### DESCRIPTION

The present invention relates to methods for preparing hydroxylated collagen peptide components and to hydroxylated collagen peptide components, in particular hydroxylated collagen peptides, obtained from these methods.

Collagen is the most abundant protein in the human body and, as the main component of the extracellular matrix, gives various tissues their characteristic flexibility and elasticity. Collagen polypeptide chains form a helical structure caused by the repetitive consensus sequence of amino acids (Gly-X-Y)ₙ, wherein “Gly” stands for glycine and “X” and “Y” each stand for any amino acid. The most common repetitive unit in collagen is Gly-Pro-Hyp, wherein “Pro” is the amino acid proline and “Hyp” is hydroxyproline ((S)-(−)-trans-4-hydroxyproline). The degree of hydroxylation of proline in human collagens varies between approx. 42-54%. In bovine collagen, the degree of hydroxylation is in the range of 45%.

Collagen is an important source of biologically active peptides with multiple applications. Due to their high antioxidant and antihypertensive activity in combination with their low antigenicity, collagen peptides are particularly suitable for use as “functional foods”. In terms of sustainability, it is desirable to prepare collagen peptides recombinantly, especially from mammals such as cattle (Bos taurus). For the recombinant production of collagen polypeptide chains of animal, in particular bovine, origin, preferably peptides or fragments from the natural sequence of a collagen polypeptide chain, in particular of α-1 type I collagen (Col1A1), different cell-based expression systems can be considered in principle: prokaryotes, such as Escherichia coli (E. coli), as well as eukaryotic organisms, such as yeasts, plants, mammalian cells and insect cells. While bacteria and yeasts do not possess natural mechanisms for hydroxylation of proline, they allow for cost-effective production of recombinant proteins.

The use of prokaryotic systems, such as E. coli, for the industrial production of recombinant collagen peptides offers the advantage that they are diversely characterised, are genetically easily accessible, have low complexity, have a high specific growth rate, can be grown to high cell densities (high cell density fermentation), grow on low-cost culture media, allow rapid expression of recombinant proteins with high expression levels, and high space-time yields can often be achieved in prokaryotic systems.

Recombinant expression of the human α-chain of type III collagen (hCol3A1) using the host organism Escherichia coli is well known from the prior art (Shi et al., Protein J., 2017, 36, 322-331; Rutschmann et al., Appl. Microbiol. Biotechnol., 2014, 98, 4445-4455). Despite the characteristic collagen sequence motif (Gly-X-Y)n, different collagens, especially collagens of different origins, sometimes have significantly different sequences and thus different properties with respect to recombinant expression in prokaryotic systems. For example, bovine Col1A1 (bCol1A1) has a sequence identity of only 62.1% and a sequence similarity of 67.5% to human Col3A1, which results in a different behaviour with regard to the expression of bovine collagen Col1A1 and means that the cytosolic expression approach described in the prior art cannot be transferred to the recombinant expression of bovine collagen Col1A1 in prokaryotic systems. The production of recombinant collagen peptides, especially collagen peptides of bovine origin, using prokaryotic systems, for example with the host organism Escherichia coli, presents a particular challenge, as collagen peptides may possess antimicrobial activity in addition to the desired biological activity. Some naturally occurring collagen peptides, such as the bovine Col1A1, have been shown to have antimicrobial activity compared to other prior art collagen peptides already successfully expressed in E. coli—such as marine collagens, collagen-like proteins of bacterial or artificial origin and so-called designer collagens, for example consisting of repetitive GEK (G: glycine, E: glutamic acid, K: lysine) and GDK sequences (D: aspartic acid) and non-naturally occurring amino acid sequences (no 100% match to natural collagen such as Col1A1)—have a higher hydrophobicity (33-43%) and/or a higher proline content (20-31%). Due to the resulting antimicrobial effect, the recombinant expression of many collagen peptides in prokaryotic systems therefore proves to be problematic.

Furthermore, a particular problem with regard to the preparation of recombinant hydroxylated collagen peptides is that prokaryotes, in contrast to many eukaryotes, are not naturally capable of hydroxylating proline either post-translationally or pre-translationally. Various approaches are already known from the prior art to enable the host organism E. coli to post-translationally hydroxylate (S)-proline (L-proline) using oxygen, 2-oxoglutarate and ascorbate to (2S,4R)-4-hydroxyproline (L-hydroxyproline). Thus, recombinant expression of human prolyl 4-hydroxalse (P4H), which is an α₂β₂-tetramer, has already been described in active form in both the cytosol and periplasm of E. coli (Kersteen et al., Protein Expr. Purif., 2004, 38, 279-291; Neubauer et al., Matrix Biol., 2005, 24, 59-68). Furthermore, human prolyl 4-hydroxylase was successfully co-expressed with artificial collagen sequences as well as a human-like collagen in the cytosol of E. coli (Pinkas et al., ACS Chem. Biol., 2011, 6, 320-324; Tang et al., Appl. Biochem. Biotechnol. 2016, 178, 1458-1470). Although human P4H could be expressed in active form in E. coli, there are no reports of recombinant expression of bovine prolyl 4-hydroxylase (P4H) in E. coli. It must be taken into account here that bovine P4H has only a low homology to human P4H. The bovine α-subunit has a sequence identity of about 37% and a sequence similarity of about 57% to the human α-subunit and the bovine β-subunit has a sequence identity of about 33% and a sequence similarity of about 50% to the human β-subunit. A problem with the recombinant expression of all P4Hs from vertebrates in prokaryotes is that only the α₂β₂-tetramer with a comparatively very high molecular weight of about 240 kDa has catalytic activity and the formation of intramolecular disulphide bridges is necessary for the formation of the native structure of the α-subunit and the association to the tetramer. In vitro association of the subunits to the tetramer does not work and co-expression of the β-subunit is necessary to keep the α-subunit in soluble form. Another problem is that the activity and stability of the tetramer is highly dependent on the availability of collagen substrates, which makes the production process and the expression/induction strategy very difficult. Due to the multimeric organisation and limited stability of animal prolyl 4-hydroxylases, coupled with their very limited activity towards short collagen substrates, efficient and correct hydroxylation of collagen peptides, in particular of bovine Col1A1 collagen peptides, using bovine prolyl 4-hydroxylase in E. coli is not guaranteed. Moreover, since productivity usually decreases with increasing numbers of different genes to be recombinantly expressed, a monomeric P4H with a preference for the collagen-typical hydroxylation of proline in the Y-position would be desirable. Bacterial P4Hs are predicted to be present in various genomes, but very few bacterial P4Hs have been characterised so far, in particular with regard to their hydroxylation pattern. An example of this is the P4H from Bacillus anthracis, which, however, hydroxylates in both the X and Y positions of the collagen motif (Gly-X-Y)ₙ (Schnicker et al., J. Biol. Chem., 2016, 291, 13360-13374) and thus cannot be considered for generating a hydroxylation pattern as identical as possible to the bovine Col1A1 .

The present invention is therefore based on the technical problem of providing a method for preparing a recombinantly produced hydroxylated collagen peptide component, in particular recombinant hydroxylated collagen peptides, which overcomes the aforementioned disadvantages, which in particular allows recombinant hydroxylated collagen peptide components, in particular recombinant hydroxylated collagen peptides, to be prepared in prokaryotic systems, even on a larger industrial and cost-effective scale. In particular, the present invention is based on the technical problem of providing a method for preparing a recombinant hydroxylated collagen peptide component with a collagen-typical hydroxylation pattern in prokaryotic systems.

The present invention solves the underlying technical problem by the subject matter of the independent claims, in particular by providing methods for preparing a recombinant hydroxylated collagen peptide component according to the present invention.

The present invention relates to a method for preparing a recombinant collagen peptide component in prokaryotic systems, comprising the steps of:
(a) providing a prokaryotic expression system comprising at least one nucleotide sequence encoding at least one recombinant collagen peptide component, wherein the at least one recombinant collagen peptide component encoding nucleotide sequence comprises the nucleotide sequence of at least one collagen peptide,
b) cultivating the prokaryotic expression system in a culture medium under conditions which allow expression of the at least one recombinant collagen peptide component to obtain at least one collagen peptide component, wherein the collagen peptide component has a reduced hydrophobicity and/or a reduced proline content relative to the at least one collagen peptide encoded by the collagen peptide component encoding nucleotide sequence,
c) recovering the collagen peptide component.

In particular, the present invention is based on the identification of the combination of the proline content and the hydrophobicity as crucial factors influencing the antimicrobial activity of some collagen peptides. Thereby, the hydrophobic character of the collagen peptides presumably leads to the incorporation of the peptides into the bacterial membranes and thus to the loss of membrane integrity, as is the case with many extracellularly applied antimicrobial peptides. The high proline content of the collagen peptides presumably leads, in analogy to the class of proline-rich antimicrobial peptides, to a disruption of cell metabolism at the level of protein synthesis without disruption of the cell membrane. The method according to the invention starts from a prokaryotic expression system, preferably E. coli, which has at least one nucleotide sequence encoding at least one recombinant collagen peptide component, wherein the at least one recombinant collagen peptide component encoding nucleotide sequence comprises the nucleotide sequence of at least one collagen peptide, that is to say that the nucleotide sequence encoding at least one recombinant collagen peptide component consists at least of a sequence encoding a collagen peptide, but additional peptide residues N- and/or C-terminal to the at least one collagen peptide may also be encoded by this nucleotide sequence, i.e. the collagen peptide component may also be a collagen fusion peptide.

In a preferred embodiment, in the nucleotide sequence encoding the collagen peptide component of the prokaryotic expression system, the nucleotide sequence encoding a collagen peptide is fused with at least one nucleotide sequence encoding at least one, preferably hydrophilic, peptide residue. The fusion of the collagen peptide with the at least one peptide residue, preferably hydrophilic peptide residue, results in the formation of an overall hydrophobicity and/or proline reduced collagen fusion peptide in the prokaryotic expression system.

According to this preferred embodiment, the collagen peptide component is a collagen fusion peptide which, in addition to the amino acid sequence of the collagen peptide, has at least one peptide residue, in particular a hydrophilic peptide residue, in particular at least one protein tag, preferably His tag, a signal peptide and/or a lamination domain.

Preferably, the at least one collagen peptide of the at least one collagen peptide component, in particular of the collagen fusion peptide, is separated from the at least one peptide residue, in particular the at least one protein tag, the at least one signal peptide and/or the at least one lamination domain by specific recognition sequences.

Particularly preferably, the specific recognition sequences are selected from the group consisting of Factor Xa (Ile-(Glu/Asp)-Gly-Arg), TEV (Glu-Asn-Leu-Tyr-Phe-Gln-(Gly/Ser)), thrombin (Leu-Val-Pro-Arg-Gly-Ser), trypsin recognition sequence, papain recognition sequence.

In another preferred embodiment, the lamination domain is an N-terminal amino acid sequence of Col1A1 procollagen from Bos taurus or a V-domain of the collagen-like protein ScI2.28 from Streptococcus pyogenes. Preferably, the N-terminal amino acid sequence of the Col1A1 procollagen from Bos taurus has the amino acid sequence according to SEQ ID No. 17, preferably consists thereof. Preferably, the V-domain of the collagen-like protein ScI2.28 from Streptococcus pyogenes has the amino acid sequence according to SEQ ID No. 18, preferably consists thereof.

In a particularly preferred embodiment, the collagen peptide component according to the present invention may be fused with one or with two peptide residues. Preferably, the collagen peptide of the collagen peptide component is fused with at least one, preferably at least two, preferably at least three, preferably at least four, peptide residues, in particular hydrophilic peptide residues.

In a particularly preferred embodiment of the present invention, the at least one peptide residue of the collagen peptide component, in particular the collagen fusion peptide, is maltose-binding protein (MBP). Preferably, MBP is fused to the N-terminus of the collagen peptide. In another preferred embodiment, MBP is fused to the C-terminus of the collagen peptide. Preferably, MBP has the amino acid sequence according to SEQ ID No. 7, preferably consists thereof.

According to a preferred embodiment of the present invention, the at least one peptide residue of the collagen peptide component, in particular of the collagen fusion peptide is superfolder green fluorescent protein (superfolder GFP). Preferably, superfolder-GFP is fused to the N-terminus of the collagen peptide. In another preferred embodiment, superfolder-GFP is fused to the C-terminus of the collagen peptide. Preferably, superfolder-GFP has the amino acid sequence according to SEQ ID No. 5, preferably consists thereof.

In a further preferred embodiment, the at least one peptide residue of the collagen peptide component, in particular of the collagen fusion peptide, is Mxe-GyrA intein having a C-terminal chitin binding domain. Preferably, Mxe-GyrA-intein with C-terminal chitin binding domain is fused to the N-terminus of the collagen peptide. In another preferred embodiment, Mxe-GyrA-Intein with C-terminal chitin binding domain is fused to the C-terminus of the collagen peptide.

Preferably, Mxe-GyrA-intein with C-terminal chitin binding domain has the amino acid sequence according to SEQ ID No. 8, preferably consists thereof.

Preferably, the at least one peptide residue of the collagen peptide component, in particular the collagen fusion peptide, is Mxe-GyrA-intein with C-terminal chitin binding domain and superfolder GFP. Preferably, Mxe-GyrA-intein with C-terminal chitin binding domain and superfolder-GFP is fused to the N-terminus of the collagen peptide. In another preferred embodiment, Mxe-GyrA-intein with C-terminal chitin binding domain and superfolder-GFP is fused to the C-terminus of the collagen peptide. Preferably, Mxe-GyrA-intein with C-terminal chitin binding domain and superfolder-GFP has the amino acid sequence according to SEQ ID No. 9, preferably consists thereof.

In a particularly preferred embodiment of the present invention, the collagen peptide of the collagen peptide component, in particular the collagen fusion peptide, is fused at the N-terminus to MBP and fused at the C-terminus to superfolder GFP, Mxe-GyrA protein with C-terminal chitin binding domain or Mxe-GyrA protein with C-terminal chitin binding domain and superfolder GFP.

In a preferred embodiment of the present invention, the collagen peptide component, in particular the collagen fusion peptide, comprises a collagen peptide and at least one N- and/or C-terminal secretion signal peptide, preferably a cleavable, in particular enzymatically cleavable, N- and/or C-terminal secretion signal peptide. Preferably, the collagen peptide component, in particular the collagen fusion peptide, comprises a collagen peptide, an N- and/or C-terminal secretion signal peptide and at least one further peptide residue, in particular at least one further hydrophilic peptide residue. Particularly preferably, the N- and/or C-terminal secretion signal peptide is selected from HlyA, HlyAc and the catalytic domain of a cellulase from Bacillus subtilis KSM-64.

Particularly preferably, the HlyA signal peptide sequence contains the amino acid sequence according to SEQ ID No. 1, preferably consists thereof.

In another preferred embodiment of the present invention, the HlyAc signal peptide sequence contains the amino acid sequence according to SEQ ID No. 2, preferably consists thereof.

Particularly preferably, the collagen peptide component is a collagen fusion peptide in which the collagen peptide is fused at the N-terminus and/or C-terminus with a hydrophilic peptide residue, in particular with superfolder GFP at the N-terminus and with an HlyA signal sequence or an HlyAc signal sequence at the C-terminus.

Preferably, the superfolder GFP has the amino acid sequence according to SEQ ID No. 5, preferably consists thereof.

Particularly preferably, the collagen peptide component is a collagen fusion peptide in which the collagen peptide at the N-terminus is fused to the catalytic domain of a cellulase from Bacillus subtilis KSM-64. Preferably, the catalytic domain of a cellulase from Bacillus subtilis KSM-64 has the amino acid sequence according to SEQ ID No. 6, preferably consists thereof. In this preferred embodiment, the collagen peptide component, in particular the collagen fusion peptide, may have, in addition to the catalytic domain of a cellulase from Bacillus subtilis KSM-64, further peptide residues fused to the N-terminus and/or C-terminus of the collagen peptide.

In a preferred embodiment, the prokaryotic expression system provided in step a) additionally comprises at least one nucleotide sequence encoding HlyB and at least one nucleotide sequence encoding HlyD and is cultivated in step b) in the culture medium under conditions that allow expression of the at least one recombinant collagen peptide component and of HlyB and HlyD. Co-expression of HlyB, HlyD and a collagen peptide component comprising the secretion signal peptide advantageously allows the collagen peptide component to be secreted into the culture medium.

Preferably, HlyB has the amino acid sequence according to SEQ ID No. 3, preferably consists thereof. In another preferred embodiment, HylD has the amino acid sequence according to SEQ ID No. 4, preferably consists thereof.

By fusing the collagen peptide of the collagen peptide component, in particular the collagen fusion peptide, with the secretion signal peptide, in particular with the signal sequence HlyA, the signal sequence HlyAc or with the catalytic domain of a cellulase from Bacillus subtilis KSM-64 according to the aforementioned embodiments of the present invention, it is advantageously possible to produce recombinant collagen peptide components, in particular collagen peptides or collagen fusion peptides, in a prokaryotic expression system and to secrete these directly into the culture medium.

In a further preferred embodiment of the present invention, the collagen peptide component, in particular the collagen fusion peptide, comprises a collagen peptide and at least one N-terminal signal peptide, preferably a cleavable, in particular enzymatically cleavable, N-terminal Sec- or TAT-specific signal peptide. In a particularly preferred embodiment of the present invention, the prokaryotic expression system is an E. coli leaky mutant. Due to the translocation of the collagen peptide component, in particular the collagen peptide or the collagen fusion peptide, into the periplasmic space mediated via the N-terminal Sec- or TAT-specific signal peptide, it is compatible with an E. coli leaky mutant, it is advantageously possible to secrete the collagen peptide component translocated into the periplasmic space, in particular the collagen fusion peptide, into the culture medium via the partially permeable outer membrane of the leaky mutant.

Advantageously, the aforementioned embodiments of the present invention can avoid having to isolate the collagen peptide component from the cytoplasm or from a periplasmic space of the prokaryotic system. In particular, it can be avoided that the collagen peptide component, in particular the collagen peptide or the collagen fusion peptide, is present intracellularly and thus with a plurality of host proteins. According to these preferred embodiments, it is advantageously also no longer necessary to partially or completely disrupt the cells (periplasmic expression: selective periplasmic disruption; cytosolic expression: complete lysis of the cell), which also avoids having to isolate the collagen peptide component from a complex protein mixture. In Particular, when expressed in the cytoplasm, there is a risk that the recombinant collagen peptide component is subject to proteolysis by intracellular proteases. By translocating the synthesised collagen peptide component into the periplasmic space of a leaky mutant or by direct secretion into the culture medium, its purification is considerably simplified and more economical. At the same time, the collagen peptide component is largely protected from proteolysis in the culture medium. Furthermore, the secretion of the collagen peptide component into the culture medium often allows higher product titres to be achieved than with cytosolic expression. Since the collagen peptide component according to these preferred embodiments is present in the culture medium largely free of host proteins, no affinity chromatographic or multi-step complex purification is required for its isolation, but only an ultra-/diafiltration step. Furthermore, the process step of cell disruption can advantageously be omitted. If necessary, before, after or during the recovery of the collagen peptide component, in particular the collagen peptide or the collagen fusion peptide, a cleavage of C- and/or N-terminal procollagen fragments for the recovery of a collagen peptide component, in particular a collagen peptide or a collagen fusion peptide, can be carried out.

In a further embodiment, it is provided that following process step b) and before method step c) or following method step c) in a method step d), cleavage, in particular enzymatic cleavage, of the at least one peptide residue from the collagen peptide component, in particular the recombinant collagen fusion peptide, takes place. According to this preferred embodiment of the present invention, the collagen peptide component obtained by the method of the invention in step b) and recovered in step c) has at least one peptide or protein sequence fused to the N- and/or C-terminus of the collagen peptide, in particular which is cleavable, preferably enzymatically cleaveable. This at least one peptide or protein sequence fused to the N- and/or C-terminus of the collagen peptide reduces the proline content and/or the hydrophobicity of the collagen fusion peptide expressed in the prokaryotic expression system during expression in the prokaryotic expression system and thus counteracts the antimicrobial activity of the collagen peptide. According to this embodiment, after step b) and before step c) or after recovering the collagen peptide component in step c), cleavage, in particular enzymatic cleavage, of the at least one peptide sequence fused to the N- and/or C-terminus of the collagen peptide is preferably performed to finally obtain an isolated collagen peptide.

According to another preferred embodiment of the present invention, in addition or alternatively to the presence of a peptide residue fused N- and/or C-terminally to the collagen peptide, it may also be provided that the recombinant collagen peptide component encoded by the nucleotide sequence of the prokaryotic expression system is formed under conditions that are post-transcriptional in process step b), either i) post-transcriptionally and pre-translationally or ii) post-transcriptionally and post-translationally, result in a reduction of the hydrophobicity and/or the proline content of the collagen peptide component encoded by the nucleotide sequence relative to the at least one collagen peptide encoded by the nucleotide sequence encoding the collagen peptide component.

Particularly preferably, in step b), the recombinant collagen peptide component encoded by the nucleotide sequence of the prokaryotic expression system is formed under conditions in which post-transcriptionally and pre-translationally, i.e. before translation or during translation of the mRNA, at least one amino acid having low hydrophobicity is incorporated into the collagen peptide component, in particular into the collagen peptide or the collagen fusion peptide, in place of a hydrophobic amino acid provided by the base triplet of the mRNA, in particular proline.

In a preferred embodiment, this can be done by using such prokaryotic expression systems that do not prepare proline, in particular a hydrophobic amino acid, but instead prepare the amino acid hydroxyproline, which has lower hydrophobicity, and incorporate it by way of translation into the collagen peptide component, in particular the collagen peptide or collagen fusion peptide, so that the hydrophobicity and/or the proline content of the collagen peptide component formed, in particular of the collagen peptide or collagen fusion peptide, is reduced relative to the at least one collagen peptide encoded by the nucleotide sequence encoding the collagen peptide component.

In a particularly preferred embodiment, a reduction in the hydrophobicity and/or proline content of the collagen peptide component, in particular the collagen peptide or the collagen fusion peptide, can be achieved by cultivating the prokaryotic expression system in a hydroxyproline-containing or hydroxyproline-enriched culture medium in step b). According to this preferred embodiment, the cultivation in step b) of the method of the invention is carried out in a hydroxyproline-containing or hydroxyproline-enriched culture medium. Particularly preferably, the hydroxyproline-containing or hydroxyproline-enriched culture medium is obtained by incubating a proline-containing or proline-enriched culture medium with at least one proline 4-hydroxylase (PIN4H).

In a preferred embodiment of the present invention, it may be provided that the prokaryotic expression system is a proline-auxotrophic host cell. In a further preferred embodiment, it may be provided that the prokaryotic expression system is a proline-auxotrophic and hydroxyproline-preparing host cell.

In a particularly preferred embodiment of the present invention, it may be provided that the prokaryotic expression system is a proline-auxotrophic host cell and the culture medium is hydroxyproline-containing or hydroxyproline-enriched, in particular wherein the hydroxyproline-containing or hydroxyproline-enriched culture medium is obtained by incubating a proline-containing or proline-enriched culture medium with at least one proline-4-hydroxylase (PIN4H).

According to the invention, the reduction of hydrophobicity and/or proline content can be achieved by using a prokaryotic expression system which, in addition to the at least one nucleotide sequence encoding at least one recombinant collagen peptide component, additionally expresses at least one nucleotide sequence encoding at least one proline 4-hydroxylase. Accordingly, the reduction of hydrophobicity and/or proline content occurs post-transcriptionally and pre-translationally using the proline 4-hydroxylase (PIN4H) (EC 1.14.11.57) expressed by the prokaryotic system. PIN4Hs convert the natural amino acid L proline using oxygen and 2 oxoglutarate to L hydroxyproline, which is recognised by proline-tRNA synthetase and incorporated instead of L proline into a growing polypeptide chain of the collagen peptide component, in particular the collagen peptide or collagen fusion peptide.

Consequently, the present invention relates more particularly to a method for preparing a recombinant hydroxylated collagen peptide component in prokaryotic systems comprising the steps of:
(i) providing a prokaryotic expression system comprising at least one nucleotide sequence encoding at least one recombinant collagen peptide component and at least one nucleotide sequence encoding at least one proline 4-hydroxylase (PIN4H), wherein the nucleotide sequence encoding at least one recombinant collagen peptide component comprises the nucleotide sequence of at least one collagen peptide,
ii) cultivating the prokaryotic expression system in a culture medium under conditions which allow expression of the at least one recombinant collagen peptide component and the at least one proline 4-hydroxylase (PIN4H) to obtain at least one hydroxylated collagen peptide component, wherein the collagen peptide component has a reduced hydrophobicity and/or a reduced proline content relative to the at least one collagen peptide encoded by the collagen peptide component encoding nucleotide sequence,
iii) recovering the hydroxylated collagen peptide component.

Particularly preferably, the proline 4-hydroxylase is of bacterial origin, in particular the proline 4-hydroxylase is a proline 4-hydroxylase from Streptomyces griseoviridis, Dactylosporangium sp., Pseudomonas stutzeri, Bordetella bronchiseptica, Bradyrhizobium japonicum, Aeromonas caviae, Janthinobacterium sp. or Achromobacter xylosoxidans. In a particularly preferred embodiment, the proline 4-hydroxylase is a monomeric proline 4-hydroxylase. In a further preferred embodiment, the proline 4-hydroxylase has an amino acid sequence selected from SEQ ID No. 10 to 13, preferably consisting of an amino acid sequence selected from SEQ ID No. 10 to 13. Particularly preferably, the proline 4-hydroxylase has the amino acid sequence according to SEQ ID No. 10, preferably consists thereof. Preferably, the proline 4-hydroxylase has the amino acid sequence according to SEQ ID No. 11, preferably consists thereof. Preferably, the proline 4-hydroxylase has the amino acid sequence according to SEQ ID No. 12, preferably consists thereof. Preferably, the proline 4-hydroxylase has the amino acid sequence according to SEQ ID No. 13, preferably consists thereof.

In a further preferred embodiment of the present invention, it may be provided that in step b) following transcription and translation, i.e. post-translationally, the hydrophobicity and/or the proline content of the expressed collagen peptide component, in particular the collagen peptide or collagen fusion peptide, is reduced by post-translational modification, in particular hydroxylations, in particular proline hydroxylations and/or glycosylations, relative to the at least one collagen peptide encoded by the nucleotide sequence encoding the collagen peptide component.

In another preferred embodiment of the present invention, the prokaryotic expression system provided in step a) additionally comprises at least one nucleotide sequence encoding at least one prolyl 4-hydroxylase and is cultivated in step b) in a culture medium under conditions, which allow expression of the at least one recombinant collagen peptide component and the at least one prolyl 4-hydroxylase, wherein the collagen peptide component has a reduced hydrophobicity and/or a reduced proline content relative to the at least one collagen peptide encoded by the collagen peptide component encoding nucleotide sequence.

Preferably, the prokaryotic expression system provided in step a) is E. coli. In another preferred embodiment of the present invention, the prokaryotic expression system provided in step a) is Bacillus subtilis.

Preferably, the at least one nucleotide sequence encoding at least one recombinant collagen peptide component of the prokaryotic expression system is a codon-optimised nucleic acid, in particular at least one nucleotide sequence encoding at least one recombinant collagen peptide component adapted to the preferred codon usage of the prokaryotic expression system.

The method according to the invention advantageously allows to prepare a recombinant collagen peptide generally toxic for the prokaryotic expression system, in particular in E. coli, with high efficiency and in high purity.

The method according to the invention is particularly advantageous in that recombinant collagen peptides can also be prepared in an industrial or large-scale process route. In a particularly preferred embodiment, the recombinant collagen peptide components provided according to the invention are biologically active. In particular, the collagen peptide components prepared by the method according to the invention show biological activity on the biosynthesis of proteins of the extracellular matrix, preferably on the biosynthesis of collagen, elastin and/or proteoglycans. Particularly preferably, the collagen peptide components produced by the method according to the invention show a biological activity on chondrocytes, fibroblasts and/or osteoblasts.

Particularly preferably, the at least one nucleotide sequence encoding at least one recombinant collagen peptide component is codon-optimised.

Preferably, the at least one collagen peptide encoded by the at least one nucleotide sequence has a amino acid sequence occurring in collagen of types I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, preferably type I, II or III, preferably type I, preferably type II, preferably type III.

Preferably, the at least one collagen peptide encoded by the at least one nucleotide sequence has an amino acid sequence occurring in collagen from vertebrates, in particular fish, amphibians, reptiles, birds and mammals, in particular in human, bovine, porcine, equine or avian collagen of types I, II or III, preferably type I, preferably type II, preferably type III.

Particularly preferably, the collagen peptide encoded by the nucleotide sequence has an amino acid sequence occurring in bovine collagen, in particular in bovine type I collagen, preferably in the α1-chain of bovine type I collagen.

Preferably, the collagen peptide, in particular the collagen peptide of the collagen peptide component or the collagen peptide of the collagen fusion peptide, has at least one amino acid sequence selected from the group consisting of SEQ ID Nos. 25, 26, 27, 28, 29, 30, 31 and 33, preferably consisting of at least one of these.

Preferably, the collagen peptide of the collagen fusion peptide encoded by the at least one nucleotide sequence is a naturally occurring collagen peptide. In another preferred embodiment of the present invention, the collagen peptide of the collagen fusion peptide encoded by the nucleotide sequence is not a naturally occurring collagen peptide. Preferably, the collagen peptide of the collagen fusion peptide encoded by the nucleotide sequence is a genetically modified collagen peptide. In a particularly preferred embodiment of the present invention, the collagen peptide of the collagen fusion peptide encoded by the nucleotide sequence is a genetically modified collagen peptide in which at least one amino acid of the amino acid sequence of a naturally occurring collagen peptide, preferably at least one non-essential amino acid, in particular Ala, Asn, Asp, Glu, Ser, of the amino acid sequence of a naturally occurring collagen peptide, has been replaced by at least one quite specific amino acid, in particular by at least one essential amino acid, in particular Ile, Leu, Lys, Met, Phe, Thr, Trp, Val, His, Cys, Tyr, particularly preferably Trp.

In a preferred embodiment of the present invention, the recombinant collagen peptide component, in particular the recombinant collagen peptide component recovered in step c), the collagen fusion peptide or the collagen peptide, in particular the collagen peptide obtained after cleavage of the at least one peptide residue, has a size of 0.18 to 110 kDa, preferably 0.18 to 100 kDa, more preferably 0, 18 to 90 kDa, preferably 0.18 to 80 kDa, preferably 0.18 to 70 kDa, preferably 0.2 to 60 kDa, preferably 0.3 to 50 kDa, preferably 0.5 to 50 kDa, preferably 0.6 to 50 kDa, preferably 0.7 to 50 kDa, preferably 0, 8 to 50 kDa, preferably 0.9 to 50 kDa, preferably 1 to 50 kDa, preferably 2 to 50 kDa, preferably 5 to 50 kDa, preferably 5 to 40 kDa, preferably 5 to 30 kDa, preferably 5 to 20 kDa, preferably 5 to 10 kDa.

In a particularly preferred embodiment of the present invention, the recombinant collagen peptide component, in particular the recombinant collagen peptide component recovered in step c), comprises the collagen fusion peptide or the collagen peptide, in particular the collagen peptide obtained after cleavage of the at least one peptide residue, has a size of 0.18 to 20 kDa, preferably 0.2 to 18 kDa, preferably 0.3 to 16 kDa, preferably 0.5 to 14 kDa, preferably 0.6 to 12 kDa, preferably 0.8 to 10 kDa, preferably 1 to 8 kDa, preferably 1 to 6 kDa, preferably 1 to 4 kDa.

In a further preferred embodiment, the recombinant collagen peptide component, in particular the recombinant collagen peptide component recovered in step c), comprises the collagen fusion peptide or the collagen peptide, in particular the collagen peptide obtained after cleavage of the at least one peptide residue, has a size of 10 to 80 kDa, preferably 10 to 70 kDa, preferably 10 to 60 kDa, preferably 10 to 50 kDa, preferably 10 to 40 kDa, preferably 10 to 30 kDa, preferably 10 to 20 kDa.

Particularly preferably, the recombinant collagen peptide component, in particular the recombinant collagen peptide component recovered in step c), the collagen fusion peptide or the collagen peptide, in particular the collagen peptide obtained after cleavage of the at least one peptide residue, is hydroxylated. In another preferred embodiment of the present invention, the recombinant collagen peptide component, in particular the recombinant collagen peptide component recovered in step c), the collagen fusion peptide or the collagen peptide, in particular the collagen peptide obtained after cleavage of the at least one peptide residue, is not hydroxylated.

In another preferred embodiment of the present invention, the recombinant collagen peptide component, in particular the recombinant collagen peptide component recovered in step c), has the collagen fusion peptide or the collagen peptide, in particular the collagen peptide obtained after cleavage of the at least one peptide residue, has a ratio of proline to hydroxyproline of 0% to 45% proline to 55% to 100% hydroxyproline (based on the number of proline and hydroxyproline residues of the collagen peptide component).

The present invention also comprises a collagen peptide component preparable, in particular prepared, by the method of the invention for preparing a recombinant collagen peptide component in prokaryotic systems.

The present invention further comprises a method for preparing a recombinant hydroxylated collagen peptide in prokaryotic systems, comprising the steps of:
aa) providing a prokaryotic expression system comprising at least one nucleotide sequence encoding at least one collagen peptide and at least one nucleotide sequence encoding at least one prolyl 4-hydroxylase,
bb) cultivating the prokaryotic expression system in a culture medium under conditions which allow expression of the at least one collagen peptide and the at least one prolyl 4-hydroxylase to obtain at least one hydroxylated collagen peptide,
cc) recovering at least one hydroxylated collagen peptide,
   wherein the at least one collagen peptide has the amino acid sequence motif (Gly-X-Y)ₙ and at least 50% of the hydroxylations in the at least one collagen peptide are at a proline in Y-position.

The method according to the invention thus advantageously allows the preparation of at least one hydroxylated collagen peptide with a hydroxylation pattern typical of collagen in prokaryotic systems.

In a preferred embodiment of the present invention, at least 50%, preferably at least 55%, preferably at least 60%, preferably at least 65%, preferably at least 70%, preferably at least 75%, preferably at least 80%, preferably at least 85%, preferably at least 90%, preferably at least 95%, preferably at least 100%, of the hydroxylations in the at least one collagen peptide are at a proline in the Y-position.

In another preferred embodiment of the present invention, the at least one collagen peptide recovered in step cc) has a degree of hydroxylation of from 5 to 100%, preferably from 5 to 90%, preferably from 5 to 80%, preferably from 10 to 70%, preferably from 15 to 60%, preferably from 20 to 50%, preferably from 30 to 50%, preferably from 35 to 50%, preferably from 40 to 50% (each based on the total number of proline and lysine residues of the collagen peptide).

Preferably, the at least one collagen peptide recovered in step cc) has a degree of hydroxylation of at least 5%, preferably at least 10%, preferably at least 15%, preferably at least 20%, preferably at least 25%, preferably at least 30%, preferably at least 35%, preferably at least 40%, preferably at least 45%, preferably at least 50% (each based on the total number of proline and lysine residues of the collagen peptide). Preferably, the at least one collagen peptide recovered in step cc) has a degree of hydroxylation of at most 80%, preferably at most 75%, preferably at most 70%, preferably at most 65%, preferably at most 60%, preferably at most 55%, preferably at most 50% (each based on the total number of proline and lysine residues of the collagen peptide).

In a particularly preferred embodiment of the present invention, the nucleotide sequence encoding at least one collagen peptide is a naturally occurring nucleotide sequence. Preferably, the nucleotide sequence encoding at least one collagen peptide is a mammalian nucleotide sequence. Preferably, the nucleotide sequence encoding at least one collagen peptide is a nucleotide sequence of bovine origin. Preferably, the nucleotide sequence encodes a naturally occurring collagen peptide.

According to another preferred embodiment of the present invention, the nucleotide sequence encoding at least one collagen peptide is not a naturally occurring nucleotide sequence. Preferably, the nucleotide sequence encoding at least one collagen peptide is a genetically modified nucleotide sequence. In another preferred embodiment of the present invention, the nucleotide sequence does not encode a naturally occurring collagen peptide. Preferably, the collagen peptide encoded by the nucleotide sequence is a genetically modified collagen peptide. In a particularly preferred embodiment of the present invention, the collagen peptide encoded by the nucleotide sequence is a genetically modified collagen peptide in which at least one amino acid of the amino acid sequence of a naturally occurring collagen peptide, preferably at least one non-essential amino acid, in particular Ala, Asn, Asp, Glu, Ser, of the amino acid sequence of a naturally occurring collagen peptide, has been replaced by at least one quite specific amino acid, in particular by at least one essential amino acid, in particular Ile, Leu, Lys, Met, Phe, Thr, Trp, Val, His, Cys, Tyr, particularly preferably Trp.

In a preferred embodiment of the present invention, the nucleotide sequence encoding at least one prolyl 4-hydroxylase is a nucleotide sequence of bacterial or plant origin. Preferably, the nucleotide sequence encoding at least one prolyl 4-hydroxylase is a nucleotide sequence of bacterial origin. In another preferred embodiment of the present invention, the at least one nucleotide sequence encoding prolyl-4-hydroxylase is a nucleotide sequence of plant origin, preferably a nucleotide sequence from Arabidopsis thaliana. Preferably, the at least one nucleotide sequence encoding prolyl 4-hydroxylase is codon-optimised from plant origin, preferably from Arabidopsis thaliana.

Particularly preferably, the at least one nucleotide sequence encoding prolyl-4-hydroxylase has the nucleotide sequence according to SEQ ID No. 14. Preferably, the at least one prolyl 4-hydroxylase-encoding nucleotide sequence has, at least 80%, preferably at least 85%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99%, sequence identity to the nucleotide sequence according to SEQ ID No. 14.

Particularly preferably, the nucleotide sequence encoding at least one prolyl 4-hydroxylase encodes at least one prolyl 4-hyroxylase from Arabidopsis thaliana, in particular at least one prolyl 4-hydroxylase comprising an amino acid sequence according to SEQ ID No. 15.

In another preferred embodiment of the present invention, the prolyl 4-hydroxylase, in particular the prolyl 4-hydroxylase from Arabidopsis thaliana, is a fusion protein. In a preferred embodiment, the prolyl 4-hydroxylase, in particular the prolyl 4-hydroxylase from Arabidopsis thaliana, is N-terminally fused to MBP. Particularly preferably, the at least one prolyl 4-hydroxylase, in particular the prolyl 4-hydroxylase from Arabidopsis thaliana, has an amino acid sequence according to SEQ ID No. 16, preferably consists thereof.

The present invention further relates to a hydroxylated collagen peptide preparable, in particular prepared, by the method of the invention for preparing a recombinant hydroxylated collagen peptide in prokaryotic systems.

The present invention further comprises a method for preparing a recombinant hydroxylated collagen peptide component in prokaryotic systems, comprising the method for preparing a recombinant collagen peptide component in prokaryotic systems according to the present invention, wherein the prokaryotic expression system provided in step a) additionally comprises at least one nucleotide sequence encoding at least one prolyl 4-hydroxylase, and the prokaryotic expression system in step b) is carried out in a culture medium under conditions that allow expression of the at least one collagen peptide and the at least one prolyl 4-hydroxylase.

Accordingly, the present invention also particularly comprises a method for preparing a recombinant hydroxylated collagen peptide component in prokaryotic systems comprising the steps of:
(i) providing a prokaryotic expression system comprising at least one nucleotide sequence encoding at least one recombinant collagen peptide component and at least one nucleotide sequence encoding at least one prolyl 4-hydroxylase, wherein the at least one recombinant collagen peptide component encoding nucleotide sequence comprises the nucleotide sequence of at least one collagen peptide,
ii) cultivating the prokaryotic expression system in a culture medium under conditions which allow expression of the at least one recombinant collagen peptide component and the at least one prolyl 4-hydroxylase to obtain at least one hydroxylated collagen peptide component, wherein the collagen peptide component has reduced hydrophobicity and/or reduced proline content relative to the at least one collagen peptide encoded by the collagen peptide component encoding nucleotide sequence,
iii) recovering the hydroxylated collagen peptide component.

In a particularly preferred embodiment of the present invention, the collagen peptide component recovered in step iii), in particular the collagen peptide or collagen fusion peptide, has the amino acid sequence motif (Gly-X-Y)ₙ, wherein at least 50% of the hydroxylations in the at least one collagen peptide are at a proline in the Y-position.

More particularly, the present invention relates to a method for preparing a recombinant hydroxylated collagen peptide component in prokaryotic systems comprising the steps of:
(a) providing a prokaryotic expression system comprising at least one nucleotide sequence encoding at least one recombinant collagen peptide component and at least one nucleotide sequence encoding at least one prolyl 4-hydroxylase, wherein the nucleotide sequence encoding at least one recombinant collagen peptide component comprises the nucleotide sequence of at least one collagen peptide,
b) cultivating the prokaryotic expression system in a culture medium under conditions which allow expression of the at least one recombinant collagen peptide component and the at least one prolyl 4-hydroxylase to obtain at least one hydroxylated collagen peptide component, wherein the collagen peptide component has reduced hydrophobicity and/or reduced proline content relative to the at least one collagen peptide encoded by the collagen peptide component encoding nucleotide sequence,
c) recovering the hydroxylated collagen peptide component,
   wherein the at least one collagen peptide has the amino acid sequence motif (Gly-X-Y)ₙ and at least 55% of the hydroxylations in the at least one collagen peptide are at a proline in the Y-position.

Accordingly, the at least one prolyl 4-hydroxylase encoded by the at least one nucleotide sequence is a prolyl 4-hydroxylase which has specificity, in particular predominant specificity, for the hydroxylation of proline residues located in the Y-position of the amino acid sequence motif (Gly-X-Y)ₙ. By the preferred, in particular predominant, hydroxylation of proline residues of the collagen peptide component, in particular of the collagen peptide or collagen fusion peptide, in the Y-position of the amino acid sequence motif (Gly-X-Y)ₙ, a hydroxylation pattern is advantageously obtained, which is similar to the hydroxylation pattern of collagen and collagen peptides of vertebrates, in particular fish, amphibians, reptiles, birds and mammals, in particular human, bovine, porcine, equine or avian collagen and collagen peptides, preferably bovine collagen and collagen peptides.

In a particularly preferred embodiment of the present invention, the collagen peptide component, in particular the collagen peptide or collagen fusion peptide, has the amino acid sequence motif (Gly-X-Y)ₙ once, preferably twice, preferably three times, preferably four times. Preferably, the collagen peptide component, in particular the collagen peptide or collagen fusion peptide, has the amino acid sequence motif (Gly-X-Y)ₙ at least once, preferably at least twice, preferably at least three times, preferably at least four times. In another preferred embodiment of the present invention, the collagen peptide component, in particular the collagen peptide or collagen fusion peptide, has the amino acid sequence motif (Gly-X-Y)ₙ at most twice, preferably at most three times, preferably at most four times.

Particularly preferably, n is an integer≥1, preferably ≥2, preferably ≥3, preferably ≥4, preferably ≥5, preferably ≥6, preferably ≥7, preferably ≥8, preferably ≥9, preferably ≥10, preferably ≥15, preferably ≥20, preferably ≥25, preferably ≥30, preferably ≥35, preferably ≥40, preferably ≥preferably ≥50.

According to the invention, it can be provided, for example, that the amino acid sequence motif (Gly-X-Y)ₙ occurs x times, for example once, twice, three times or four times, in the amino acid sequence of the collagen peptide component, in particular of the collagen peptide or collagen fusion peptide, where n is in each case an integer≥1, preferably ≥2, preferably ≥3, preferably ≥4, preferably ≥5, preferably ≥6, preferably ≥7, preferably ≥8, preferably ≥9, preferably ≥10, preferably ≥15, preferably ≥20, preferably ≥25, preferably ≥30, preferably ≥35, preferably ≥preferably ≥45, preferably ≥50.

The present invention also relates to a hydroxylated collagen peptide component preparable, in particular prepared, by one of the methods of the invention for preparing a recombinant hydroxylated collagen peptide component in prokaryotic systems.

The statements and embodiments made above in connection with the method according to the invention for preparing a recombinant collagen peptide component in prokaryotic systems also apply mutatis mutandis to the method for preparing a recombinant hydroxylated collagen peptide in prokaryotic systems and the methods for preparing a recombinant hydroxylated collagen peptide component in prokaryotic systems, and vice versa.

In the context of the present invention, the term “collagen” is understood in a manner customary in the art, in particular as defined for example in WO 01/34646. In a preferred embodiment, the term “collagen” relates to collagen types I to XXVII. In a further preferred embodiment, the term “collagen” is understood to mean a peptide having the sequence glycine-proline, glycine-4-hydroxyproline or glycine-X-4-hydroxyproline, preferably the repetitive motif (Gly-X-Y)ₙ, wherein X and Y can be any amino acid, preferably proline and 4-hydroxylproline. Particularly preferably, the term “collagen” is understood to mean a peptide having the repetitive motif (Gly- Pro-Y)ₙ and/or (Gly-X-Hyp)ₘ, wherein X and Y can be any amino acid.

In the context of the present invention, the term “collagen peptide” is understood to mean a protein or peptide having an amino acid sequence occurring in collagen as defined above, wherein the protein or peptide is at least a dipeptide, preferably an oligopeptide or polypeptide. In particular, the collagen peptide may be present in chemically modified form, in particular hydroxylated and/or glycosylated form, or may be unmodified.

A “collagen peptide” within the meaning of the present invention may also be a collagen protein. In particular, the collagen peptide of the present invention may be in single-stranded form, but the collagen peptide of the present invention may also be present as a di- or trimer, in particular a trimer, of the same or different collagen peptides, in particular also as a tripelhelical collagen peptide.

In the context of the present invention, a “naturally occurring collagen peptide” is understood to be a collagen peptide that can be isolated directly from natural sources, i.e. a collagen peptide that has an amino acid sequence as encoded in naturally occurring nucleotide sequences of an organism, in particular without mutations occurring in these nucleotide sequences, in particular mutations that lead to one or more amino acid exchanges. In particular, naturally occurring collagen peptides are understood to occur naturally in a vertebrate, in particular in cattle, or an invertebrate, in particular a jellyfish. In a particularly preferred embodiment, a naturally occurring collagen peptide is a collagen peptide that occurs in cattle.

According to the invention, the term “collagen peptide component” means a peptide comprising at least the amino acid sequence of a collagen peptide. In a preferred embodiment, the collagen peptide component may comprise the amino acid sequence of the collagen peptide, i.e. be a collagen peptide. However, in a further embodiment, it is also conceivable that the collagen peptide component comprises the amino acid sequence of at least one collagen peptide and at least one further amino acid sequence which is not a collagen peptide. According to this preferred embodiment, the collagen peptide component is a collagen fusion peptide. Preferably, the collagen peptide component, in particular the collagen fusion peptide, has at least one peptide residue, in particular hydrophilic peptide residue, fused N- and/or C-terminally to the amino acid sequence of the collagen peptide.

According to the invention, a “host cell” is understood to be a living cell capable of expressing peptides or proteins encoded in foreign DNA, in particular in recombinant DNA.

In the context of the present invention, a “recombinant collagen peptide” is understood to mean a collagen peptide recovered by biotechnological recombinant preparation using an expression system. In accordance with the invention, it is inherent in the “recombinant collagen peptide” that they are not recovered from natural sources.

In the context of the present invention, a “nucleotide sequence” is understood to mean the sequence of nucleotides of a nucleic acid, in particular a polynucleic acid strand, in particular a DNA strand. A “nucleotide sequence” is therefore to be understood both as an informational unit and as the DNA strand physically manifesting this information.

In the context of the present invention, the terms “codon-optimisation” and “codon-optimised” are understood to mean the adaptation of the nucleotide sequence, in particular of the base triplets coding for an amino acid, to the base triplets coding for a particular amino acid preferably used by the selected expression system, in particular the selected prokaryotic expression system. “Codon optimisation” is based on the fact that the different base triplets coding for a particular amino acid are used with different frequency by different species during protein biosynthesis. Accordingly, “codon optimisation” leads to a change in the nucleic acid coding for a particular amino acid sequence, but not to a change in the coded amino acid sequence itself.

According to the invention, a “reduction in hydrophobicity” is understood to be a reduction in the overall hydrophobicity of the collagen peptide component in question relative to the overall hydrophobicity of the collagen peptide of the collagen peptide component. According to the invention, the “reduction in hydrophobicity” can be achieved, for example, by fusion of the collagen peptide with at least one peptide residue, preferably with at least one hydrophilic peptide residue. Furthermore, the hydrophobicity of the collagen peptide component can be reduced, for example, by pre-translational or post-translational hydroxylation of proline to hydroxyproline. The hydrophobicity of the peptide can be determined, for example, using the Eisenberg Consensus Scale (ECS), the Gunnar von Heijne Scale, the Kyte and Doolittle Scale, the Wimley-White Scale, the aWW Scale, the Engelman-Steitz Scale or other determination methods known to the skilled person. Preferably, the reduction in the total hydrophobicity of the collagen peptide component in question relative to the total hydrophobicity of the collagen peptide of the collagen peptide component is at least 5%, preferably at least 10%, preferably at least 20%, preferably at least 30%, preferably at least 40%, preferably at least 50%, preferably at least 60%, preferably at least 70%, preferably at least 80%.

According to the invention, the term “reduction in proline content” denotes a reduction in the percentage of proline residues in the total number of amino acids in the collagen peptide component in question relative to the percentage of proline residues in the total number of amino acids in the at least one collagen peptide encoded by the nucleotide sequence encoding the collagen peptide component. According to the invention, a “reduction in proline content” of the collagen peptide component can be achieved, for example, by the collagen peptide component being a collagen fusion peptide. By fusing with at least one peptide residue, the percentage of proline residues in the collagen peptide component is reduced compared to the percentage in the proline- rich collagen peptide. Another way to “reduce the proline content” is to pre- or post-translationally hydroxylate proline to hydroxyproline. Preferably, the reduction in the percentage of proline residues to the total number of amino acids in the collagen peptide component in question relative to the percentage of proline residues to the total number of amino acids in the at least one collagen peptide encoded by the collagen peptide component encoding nucleotide sequence is at least 5%, preferably at least 10%, preferably at least 20%, preferably at least 30%, preferably at least 40%, preferably at least 50%, preferably at least 60%, preferably at least 70%, preferably at least 80%.

According to the invention, the term “degree of hydroxylation” refers to the percentage of hydroxylated proline and lysine residues out of the total number of proline and lysine residues of the collagen peptide component, in particular the collagen peptide or the collagen fusion peptide.

In the context of the present invention, an “expression system” is understood to mean a system in which targeted and controlled protein biosynthesis can occur. According to the invention, a “prokaryotic expression system” is to be understood as a cell-based biological system which is capable of carrying out protein biosynthesis in a targeted and controlled manner, i.e. synthesising peptides or proteins starting from a nucleic acid as information carrier.

In the context of the present invention, “expression” of a nucleotide sequence comprises in particular transcription into mRNA (messenger RNA) and subsequent translation into a peptide or, synonymously in the context of the present invention, protein.

In “conditions allowing expression of the at least one recombinant collagen peptide component to obtain at least one collagen peptide component”, “conditions allowing expression of the at least one collagen peptide and the at least one prolyl 4-hydroxylase to obtain at least one hydroxylated collagen peptide”, “conditions allowing expression of the at least one recombinant collagen peptide component and the at least one prolyl 4-hydroxylase to obtain at least one hydroxylated collagen peptide component” and “conditions allowing expression of the at least one recombinant collagen peptide component and the at least one proline 4-hydroxylase (PIN4H) to obtain at least one hydroxylated collagen peptide component” are understood according to the invention to mean conditions, such as in particular temperature, pressure, time, light and the presence or absence of inducers and/or repressors, which activate or enhance expression of the peptide and/or protein in question. In a preferred embodiment, the expression of the peptide and/or protein in question takes place in the context of a high-cell-density fermentation, in particular under high pressure, preferably high air pressure. The specific conditions enabling expression of the peptide and/or protein in question are known to the skilled person and depend on the expression system used and the expression cassette used, in particular the promoter contained therein. Expression of the peptide and/or protein in question may be constitutive or inducible, depending on the structure of the expression cassette.

In the context of the present invention, “recovering a recombinant collagen fusion peptide”, “recovering a recombinant collagen peptide”, “recovering a hydroxylated recombinant collagen peptide” or “recovering a hydroxylated collagen peptide component” means isolating a collagen peptide component, a collagen peptide or a collagen fusion peptide from a composition containing multiple components by known isolation methods, such as centrifugation methods, in particular differential centrifugation and/or density gradient centrifugation, chromatographic methods, in particular gel filtration, ion exchange, affinity and/or high performance liquid chromatography, electrophoresis methods, filtration methods and/or extraction methods, wherein enrichment and purification of the component in question from the multi-component composition can preferably be achieved by sequential application of several isolation methods.

In the context of the present invention, the terms “comprising” and “having” are understood to mean that in addition to the elements explicitly covered by these terms, further elements not explicitly mentioned may be added. In the context of the present invention, it is also understood by these terms that only the explicitly mentioned elements are covered and that no further elements are present. In this particular embodiment, the meaning of the terms “comprising” and “having” is synonymous with the term “consisting of”. In addition, the terms “comprising” and “having” also cover compositions which, in addition to the explicitly mentioned elements, also comprise further elements which are not mentioned but which are of a functionally and qualitatively subordinate nature. In this embodiment, the terms “comprising” and “having” are synonymous with the term “consisting essentially of”.

Where, in the context of the present invention, the first and second decimal places or the second decimal place are/is not indicated, they/it are/is to be set as 0.

In the context of the present invention, the term “and/or” is understood to mean that all members of a group which are connected by the term “and/or” are disclosed both alternatively to each other and cumulatively to each other in any combination. This means for the expression “A, B and/or C” that the following disclosure is to be understood thereunder: a) A or B or C or b) (A and B) or c) (A and C) or d) (B and C) or e) (A and B and C).

Further preferred embodiments of the present invention are apparent from the following aspects and from the sub-claims.

Aspect 1: A method for preparing a recombinant collagen peptide component in prokaryotic systems comprising the steps of:
a) providing a prokaryotic expression system comprising at least one nucleotide sequence encoding at least one recombinant collagen peptide component, wherein the at least one recombinant collagen peptide component encoding nucleotide sequence comprises the nucleotide sequence of at least one collagen peptide,
b) cultivating the prokaryotic expression system in a culture medium under conditions which allow expression of the at least one recombinant collagen peptide component to obtain at least one collagen peptide component, wherein the collagen peptide component has reduced hydrophobicity and/or reduced proline content relative to the at least one collagen peptide encoded by the collagen peptide component encoding nucleotide sequence,
c) recovering the collagen peptide component.

Aspect 2: The method according to aspect 1, wherein in the nucleotide sequence encoding the collagen peptide component of the prokaryotic expression system, the nucleotide sequence encoding a collagen peptide is fused to at least one nucleotide sequence encoding a peptide residue.

Aspect 3: The method of aspect 2, wherein the peptide residue has at least one of a protein tag, a signal peptide, and/or a lamination domain.

Aspect 4: The method according to any one of aspects 2 and 3, wherein the peptide residue is cleavable, in particular enzymatically cleavable.

Aspect 5: The method according to any one of aspects 2 to 4, wherein after step c) or after step b) and before step c), cleavage of the at least one peptide residue of the collagen peptide component takes place.

Aspect 6: The method according to any one of aspects 2 to 5, wherein the collagen peptide component comprises a collagen peptide and at least one N- and/or C-terminal secretion signal peptide.

Aspect 7: The method according to any one of aspects 1 to 6, wherein the collagen peptide encoded by the nucleotide sequence has an amino acid sequence occurring in bovine collagen, in particular in bovine type I collagen, preferably in the al-chain of bovine type I collagen.

Aspect 8: A method for preparing a recombinant hydroxylated collagen peptide in prokaryotic systems comprising the steps of:
aa) providing a prokaryotic expression system comprising at least one nucleotide sequence encoding at least one collagen peptide and at least one nucleotide sequence encoding at least one prolyl 4-hydroxylase,
bb) cultivating the prokaryotic expression system in a culture medium under conditions allowing expression of the at least one collagen peptide and the at least one prolyl 4-hydroxylase to obtain at least one hydroxylated collagen peptide,
cc) recovering at least one hydroxylated collagen peptide,
   wherein the at least one collagen peptide has the amino acid sequence motif (Gly-X-Y)ₙ and at least 50% of the hydroxylations in the at least one collagen peptide are at a proline in the Y-position.

Aspect 9: The method according to aspect 8, wherein at least 80% of the hydroxylations in the at least one collagen peptide are at a proline in the Y-position.

Aspect 10: The method according to aspect 8 or 9, wherein the at least one collagen peptide recovered in step cc) has a degree of hydroxylation of at least 5% (based on the total number of proline and lysine residues of the collagen peptide).

Aspect 11: The method according to any one of aspects 8 to 10, wherein the at least one nucleotide sequence encoding prolyl 4-hydroxylase is a nucleotide sequence of bacterial or plant origin.

Aspect 12: The method of aspect 11, wherein the nucleotide sequence encoding prolyl 4-hydroxylase is a nucleotide sequence from Arabidopsis thaliana.

Aspect 13: A method for preparing a recombinant hydroxylated collagen peptide component in prokaryotic systems comprising the steps of:
i) providing a prokaryotic expression system comprising at least one nucleotide sequence encoding at least one recombinant collagen peptide component and at least one nucleotide sequence encoding at least one prolyl 4-hydroxylase, wherein the nucleotide sequence encoding at least one recombinant collagen peptide component comprises the nucleotide sequence of at least one collagen peptide,
ii) cultivating the prokaryotic expression system in a culture medium under conditions which allow expression of the at least one recombinant collagen peptide component and the at least one prolyl 4-hydroxylase to obtain at least one hydroxylated collagen peptide component, wherein the collagen peptide component has reduced hydrophobicity and/or reduced proline content relative to the at least one collagen peptide encoded by the collagen peptide component encoding nucleotide sequence,
iii) recovering the hydroxylated collagen peptide component.

Aspect 14: The method according to aspect 13, wherein the collagen peptide component recovered in step iii), in particular the collagen peptide or collagen fusion peptide, has the amino acid sequence motif (Gly-X-Y)ₙ and at least 50% of the hydroxylations in the at least one collagen peptide are at a proline in the Y-position.

Aspect 15: A collagen peptide component prepared by a method according to any one of aspects 1 to 7.

Aspect 16: A hydroxylated collagen peptide prepared by a method according to any one of aspects 8 to 12.

Aspect 17: A hydroxylated collagen peptide component prepared by the method according to any one of Aspects 13 and 14.

Without limiting the general idea of the invention, the invention is illustrated below with reference to examples of embodiments.

### EXAMPLE 1

### Cytosolic Expression of a Collagen Peptide Component in E. coli

### 1. Transformation of the Cells

For each expression, 1 μL of vector pMAL-c5x (50-100 ng/μL) comprising a tetracycline resistance gene and one of SEQ ID Nos. 19 and 20, is added to 50 μL of thawed on ice, chemically competent E. coli BL21 and kept on ice for 30 mM. This is followed by heat shock for 30 s at 42° C., after which the cells are directly cooled on ice for 5 mM before 950 μL of SOC medium (20 g/L soy peptone, 5 g/L yeast extract, 0.6 g/L NaCl, 0.2 g/L KCl, 10 mL/L 1 M MgCl₂, 10 mL/L 1 M MgSO₂, 10 mL/L 2 M glucose) is added to the cells. The cells are cultivated in an overhead shaker at 37° C. for approx. 70 mM. The cells are then centrifuged at 7,000 g for 2 min at 4° C. The supernatant is discarded except for about 100 μL in which the cell pellet is resuspended. The cells are then spread on an LB agar plate prewarmed at 37° C. with 25 mg/L tetracycline and incubated at 37° C. overnight.

### 2. Cultivation and Induction of Expression

The next day, a single colony is picked and used to inoculate 30 mL of LB medium (10 g/L NaCl, 10 g/L soya peptone, 5 g/L yeast extract, pH 7.4) with 25 mg/L tetracycline in a 300 mL shake flask with baffles. The culture is cultivated at 37° C. and 120 rpm (deflection 25 mm) on an orbital shaker overnight.

The following day, 200 mL of TB medium (4 g/L glycerol, 12 g/L soya peptone, 24 g/L yeast extract, 2.31 g/L KH₂PO₄, 12.54 g/L K₂HPO₄, pH 7, 4) inoculated with 25 mg/L tetracycline in a 1 L shake flask with baffles using the preculture to an optical density of OD600=0.1 and cultivated on an orbital shaker at 37° C. and 120 rpm (deflection 25 mm). For expression in the shake flask, induce with 0.1 mM IPTG at an optical density of OD600=2-3 and continue cultivation at 28° C. before stopping cultivation after 20-22 h by harvesting cells.

If the expression did not take place in the shake flask but only in the fermenter, this previously prepared culture is not induced and cultivated overnight at 37° C. and used as the preculture 2nd stage.

The next day, a bioreactor with 12 L TB medium and 25 mg/L tetracycline is set to an OD600 of approx. 0.1 with the preculture 2nd stage. The stirrer rotation frequency starts at 200 rpm and increases according to a stirring cascade when the relative pO₂ value falls below 60% by 2% each time until the maximum stirrer rotation frequency of 1500 rpm is reached. For gassing, 12 standard litres of air per minute are used and a pressure of 0.2 bar is set. The pH is kept constant at 7.0 with 10% phosphoric acid and 4 M sodium hydroxide solution. Cultivation is carried out at 37° C. At an optical density of OD600=9.0-10.0, 0.1 mM IPTG is induced. Cultivation is terminated after stationary phase is reached.

### EXAMPLE 2

### Secretory Expression of a Collagen Peptide Component in E. coli Using the Signal Peptide HlyA

### 1. Transformation of the Cells

For each expression, 1 μL of the vector pBR322, comprising a tetracycline resistance gene and one of SEQ ID No. 21, for the collagen peptide components under investigation (50-100 ng/μL) and 1 μL of the pACYC_HlyB+D (50-100 ng/μL) for bicistronic expression of the pore proteins are added to 50 μL of chemically competent E. coli BL21(DE3) thawed on ice and kept on ice for mM. This is followed by heat shock for 30 s at 42° C., after which the cells are directly cooled on ice for 5 mM before 950 μL of SOC medium (20 g/L soy peptone, 5 g/L yeast extract, 0.6 g/L NaCl, 0.2 g/L KCl, 10 mL/L 1 M MgCl₂, 10 mL/L 1 M MgSO₂, 10 mL/L 2 M glucose) is added to the cells. The cells are cultivated in an overhead shaker at 37° C. for approx. 70 mM. The cells are then centrifuged at 7,000 g for 2 min at 4° C. The supernatant is discarded except for about 100 μL in which the cell pellet is resuspended. The cells are then spread on an LB agar plate prewarmed at 37° C. with 20 mg/L tetracycline and 20 mg/L chloramphenicol and incubated overnight at 37° C.

### 2. Cultivation and Induction of Expression

The next day, a single colony is picked and used to inoculate 30 mL of LB medium (10 g/L NaCl, 10 g/L soya peptone, 5 g/L yeast extract, pH 7.4) with 20 mg/L tetracycline and 20 mg/L chloramphenicol in a 300 mL shake flask with baffles. The culture is cultivated at 37° C. and 120 rpm (deflection 25 mm) on an orbital shaker overnight.

The following day, 200 mL of TB medium (4 g/L glycerol, 12 g/L soya peptone, 24 g/L yeast extract, 2.31 g/L KH₂PO₄, 12.54 g/L K₂HPO₄, pH 7, 4) inoculated with 25 mg/L tetracycline in a 1 L shake flask with baffles using the preculture to an optical density of OD600=0.1 and cultivated on an orbital shaker at 37° C. and 120 rpm (deflection 25 mm). For expression in the shake flask, induce with 0.1 mM IPTG at an optical density of OD600=2-3 and continue cultivation at 37° C. before stopping cultivation after 20-22 h by harvesting cells.

### EXAMPLE 3

### Secretory Expression of a Collagen Peptide Component in E. coli Using the Catalytic Domain of a Cellulase from Bacillus subtilis KSM-64

### 1. Transformation of the Cells

For each expression, 1 μL of vector pET28 (50-100 ng/μL) containing a pBR322-ori comprising a kanamycin resistance gene and one of SEQ ID Nos. 22 is added to 50 μL of thawed on ice chemically competent E. coli BL21(DE3) and kept on ice for 30 min. This is followed by heat shock for 30 s at 42° C., after which the cells are directly cooled on ice for 5 min before 950 μL of SOC medium (20 g/L soy peptone, 5 g/L yeast extract, 0.6 g/L NaCl, 0.2 g/L KCl, 10 mL/L 1 M MgCl₂, 10 mL/L 1 M MgSO₂, 10 mL/L 2 M glucose) is added to the cells. The cells are cultivated in an overhead shaker at 37° C. for approx. 70 min The cells are then centrifuged at 7,000 g for 2 min at 4° C. The supernatant is discarded except for about 100 μL in which the cell pellet is resuspended. The cells are then spread on an LB agar plate prewarmed at 37° C. with 100 mg/L kanamycin and incubated at 37° C. overnight.

### 2. Cultivation and Induction of Expression

The next day, a single colony is picked and used to inoculate 30 mL of LB medium (10 g/L NaCl, 10 g/L soya peptone, 5 g/L yeast extract, pH 7.4) with 100 mg/L kanamycin in a 300 mL shake flask with baffles. The culture is cultivated at 37° C. and 120 rpm (deflection 25 mm) on an orbital shaker overnight.

The following day, 200 mL of TB medium (4 g/L glycerol, 12 g/L soya peptone, 24 g/L yeast extract, 2.31 g/L KH₂PO₄, 12.54 g/L K₂HPO₄, pH 7, 4) inoculated with 100 mg/L kanamycin in a 1 L shake flask with baffles using the preculture to an optical density of OD600=0.1 and cultivated at 37° C. and 120 rpm (deflection 25 mm) on an orbital shaker. For expression in the shake flask, induce with 0.1 mM IPTG at an optical density of OD600=2-3 and continue cultivation at 37° C. before stopping cultivation after 20-22 h by harvesting cells.

If the expression does not take place in the shake flask but only in the fermenter, this previously prepared culture is not induced and cultivated overnight at 37° C. and used as the preculture 2nd stage.

The next day, a bioreactor with 12 L TB medium (4 g/L glycerol, 12 g/L soya peptone, 24 g/L yeast extract, 2.31 g/L KH₂PO₄, 12.54 g/L K₂HPO₄, pH 7.4) and 100 mg/L kanamycin is set to an OD600 =of approx. 0.1 with the preculture 2nd stage. The stirrer rotation frequency starts at 200 rpm and increases according to a stirring cascade when the relative pO₂ value falls below 60% by 2% each time until the maximum stirrer rotation frequency of 1500 rpm is reached. When the relative pO₂ value fell below 30%, the feed was fed (a total of 3 L volume consisting of 600 g/L glycerol, 90 g/L yeast extract, 2 g/L MgCl₂·7H₂O). For gassing, 12 standard litres of air per minute are used and a pressure of 0.2 bar is set. The pH is kept constant at 7.0 with 10% phosphoric acid and 4 M sodium hydroxide solution. Cultivation is carried out at 37° C. At an optical density of OD600=9.0-10.0, IPTG is induced with 0.1 mM. Cultivation is terminated after stationary phase is reached.

### EXAMPLE 4

### Recovery of Hydroxylated Recombinant Collagen Peptide Components Using E. coli

### 1. Post-Translational Proline Hydroxylation

For post-translation hydroxylation of collagen peptide components, in different approaches the vector pACYCDuet-1, comprising a chloramphenicol resistance gene and SEQ ID No. 23, or the vector pCDFDuet-1, comprising a streptomycin resistance gene and SEQ ID No. 23, were transformed into E. coli cells expressing a collagen peptide component.

Depending on the collagen peptide component selected, both cytosolic expression of hydroxylated collagen components (cf. example 1) and secretory expression of hydroxylated collagen components (cf. examples 2 and 3) by E. coli cells can be realised by co-expression with a prolyl 4-hydroxylase, such as prolyl 4-hydroxylase 1 from Arabidopsis thaliana, which preferably hydroxylates in the Y-position of the collagen motif (Gly-X-Y)ₙ. The post-translational hydroxylation of proline was detected by mass spectrometry.

### 1.1 Determination of the Degree of Hydroxylation

The determination of the degree of hydroxylation was based on the hydrolysis of the protein sample to be analysed into the individual amino acids, their derivatisation (AQC reagent) and subsequent separation by analytical high-performance liquid chromatography (HPLC). The degree of hydroxylation was then calculated from the chromatogram with the aid of a proline and hydroxyproline standard from the peak areas of proline and hydroxyproline from the sample to be analysed, whereby the degree of hydroxylation is the proportion of hydroxylated proline residues (hydroxyproline) related to the molar sum of all prolines (hydroxyproline and proline) (mol hydroxyproline/(mol hydroxyproline+mol proline).

The purified collagen peptides to be analysed were hydrolysed for 24 h at 110° C. in 6 M hydrochloric acid (final concentration) at a protein loading of 10 g/L in closed reaction vessels.

After cooling on ice, dropwise neutralisation on ice with sample-identical volume of 6 M sodium hydroxide solution, centrifugation of the samples for 5 min at 13,000 min⁻¹ in a microlitre centrifuge and, if necessary, dilution of the samples with 0.2 M sodium borate buffer pH 9.0, derivatisation was carried out. For this purpose, 100 μL sample was mixed with 700 μL 0.2 M sodium borate buffer pH 9.0 and 200 μL AQC reagent (2 mg/mL 6-aminoqinolyl-N-hydroxysuccinimidyl carbamate dissolved in acetonitrile (p.A.) at 55° C.) in closed HPLC vessels and incubated in a water bath at 55° C. for 10 minutes. Subsequently, analytical separation was performed by high-performance liquid chromatography (Knauer 250 mm×4 mm; Eurospher II 1005 C18P; column temperature: 37° C.; sample loop: 20 μL; volume flow: 0.8 to 1.0 mL/min; fluorescence detector (Shimadzu RF-551)): 250 nm/395 nm; Gain: x32; Sensitivity: low) using the following buffer gradient of buffer A (95% (v/v) 0.3 M sodium acetate pH 6.5+5% (v/v) acetonitrile) and buffer B (20% (v/v) acetonitrile+60% (v/v) methanol+20% (v/v).

1.2 Degree of Hydroxylation after Post-Translational Hydroxylation

Post-translational hydroxylation by prolyl 4-hydroxylase (P4H) can be assumed to hydroxylate, to a large extent specifically, only proline residues in the recombinant collagen sequence. Consequently, any protein impurities contained in the sample contribute exclusively to the measured peak area of the proline peak and thus increase the denominator in the calculation of the degree of hydroxylation. Consequently, protein impurities lead to a decrease in the measured degree of hydroxylation, which would be higher in relation to highly pure collagen peptide. The determined degrees of hydroxylation of the collagen peptides therefore represent minimum values in the case of post-translational hydroxylation.

1.3 Post-Translational Hydroxylation of a CD-Cel-TEV-10 Fusion Peptide using At-P4H

When co-expressing the 10 kDa collagen fragment in fusion with the catalytic domain of cellulase from Bacillus subtilis KSM-64 (CD-Cel-TEV-10er; see example 3) and P4H from Arabidopsis thaliana (At-P4H), a degree of hydroxylation in the range of at least 22-26% could be reproducibly measured in a feed procedure followed by Ni-NTA affinity chromatography. The measured degree of hydroxylation refers to the fusion protein, which in its truncated form contains 41 proline residues, of which 15 proline residues are contained in the truncated cellulase domain and 26 proline residues in the collagen fragment. Since it can be assumed that the 15 proline residues of the cellulase domain are not hydroxylated by the co-expressed At-P4H, because this part does not correspond to the natural At-P4H substrate spectrum, the actual degree of hydroxylation of the recombinant collagen sequence is higher than the degree of hydroxylation of the fusion protein.

### 2. Pre-Translational Proline Hydroxylation

For pre-translational hydroxylation of collagen peptide components, collagen peptide component expressing E. coli cells were transformed with a vector comprising SEQ ID No. 24 to achieve co-expression of collagen peptide components to be hydroxylated and a proline 4-hydroxylase (PIN4H) from Streptomyces griseoviridis. The incorporation of hydroxyproline into the expressed collagen peptide components was detected by mass spectroscopy.

### 2.1 Determination of the Degree of Hydroxylation

The determination of the degree of hydroxylation was performed as described in paragraph 1.1 above.

2.2 Degree of Hydroxylation after Pre-Translational Hydroxylation

In contrast to post-translational hydroxylation, the pre-translational hydroxylation approach using a proline 4-hydroxylase (PIN4H) is non-specific, i.e. the incorporation of hydroxyproline at a proline site during translation is random and essentially depends on the availability of hydroxyproline-loaded tRNA. Since the co-expression of PIN4H and collagen peptides only occurs after the build-up of sufficient protein-containing biomass during fermentation, it can be assumed that a certain proportion of host proteins, which may be present as contaminants in the protein sample to be analysed, are non-hydroxylated and the determined degree of hydroxylation is lower than would be the case if a highly pure collagen peptide were present. Therefore, it can be assumed that the degrees of hydroxylation determined in the case of post-translational hydroxylation represent minimum values.

2.3 Pre-Translational Hydroxylation of a CD-Cel-TEV-10 Fusion Peptide using Sg-PIN4H

Co-expression of the 10 kDa collagen fragment in fusion with the catalytic domain of cellulase from Bacillus subtilis KSM-64 (CD-Cel-TEV-10er; see Example 3) and PIN4H from Streptomyces griseoviridis (Sg-PIN4H) (SEQ ID No. 10) resulted in a hydroxylation degree of the fusion peptide in the range of at least 10-14% in a feed procedure followed by Ni-NTA affinity chromatography.

### EXAMPLE 5

### Cytosolic Expression of a Hydroxylated Collagen Peptide Component in E. coli

### 1. Transformation of the Cells

For each expression, 1 μL of the vector pET-28a(+) vector (50-100 ng/μL) comprising a kanamycin resistance gene and a nucleic acid sequence according to SEQ ID No. 32 encoding the collagen peptide according to SEQ ID No. 33, and 1 μL of an expression plasmid having either an At-P4H coding sequence (pACYCDuet-1) or an Sg-PIN4H coding sequence (pCDFDuet-1), to 50 μL of chemically competent E. coli BL21 thawed on ice and kept on ice for 30 min. This is followed by heat shock for 30 s at 42° C., after which the cells are directly cooled on ice for 5 min before 950 μL of SOC medium (20 g/L soy peptone, 5 g/L yeast extract, 0.6 g/L NaCl, 0.2 g/L KCl, 10 mL/L 1 M MgCl₂, 10 mL/L 1 M MgSO₂, 10 mL/L 2 M glucose) is added to the cells. The cells are cultivated in an overhead shaker at 37° C. for approx. 70 min. The cells are then centrifuged at 7,000 g for 2 min at 4° C. The supernatant is discarded except for about 100 μL in which the cell pellet is resuspended. The cells are then spread on an LB agar plate prewarmed at 37° C. with 80 mg/L kanamycin and 20 mg/L chloramphenicol (P4H) or 20 mg/L streptomycin (PIN4H), respectively, and incubated overnight at 37° C.

### 2. Cultivation and Induction of Expression

The next day, a single colony is picked and used to inoculate 30 mL LB medium (10 g/L NaCl, 10 g/L soy peptone, 5 g/L yeast extract, pH 7.4) with 80 mg/L kanamycin and 20 mg/L chloramphenicol (P4H) or 20 mg/L streptomycin (PIN4H), respectively, in a 300 mL shake flask with baffles. The culture is cultivated at 37° C. and 120 rpm (deflection 25 mm) on an orbital shaker overnight.

The following day, 200 mL of TB medium (4 g/L glycerol, 12 g/L soya peptone, 24 g/L yeast extract, 2.31 g/L KH₂PO₄, 12.54 g/L K₂HPO₄, pH 7, 4) inoculated with 80 mg/L kanamycin and 20 mg/L chloramphenicol (P4H) or 20 mg/L streptomycin (PIN4H), respectively, in a 1 L shake flask with baffles using the preculture to an optical density of OD600=0.1 and cultivated on an orbital shaker at 37° C. and 120 rpm (deflection 25 mm). For expression in the shake flask, induce with 0.1 mM IPTG at an optical density of OD600=2-3 and continue cultivation at 37° C. before stopping cultivation after 20-22 h by harvesting cells.

If the expression did not take place in the shake flask, but only in the fermenter, this previously prepared culture is not induced and cultivated overnight at 37° C. and used as a pre-culture.

The next day, a bioreactor (19 L NLF, Bioengineering AG) with 12 L TB medium and 80 mg/L kanamycin and 20 mg/L chloramphenicol (P4H) or 20 mg/L streptomycin (PIN4H) is adjusted to an OD600 of approx. 0.1 with the preculture. The stirrer rotation frequency starts at 200 rpm and increases according to a stirring cascade by 2% each time the relative pO₂ value falls below 30%. The maximum stirrer rotation frequency of 1500 revolutions per minute is not reached. When the relative pO₂ value of 60% was exceeded, the feed was fed until the relative pO₂ value was again below 60% (a total of 3 L volume consisting of 600 g/L glycerol, 90 g/L yeast extract, 2 g/L MgCl₂·7H₂O). For gassing, 15 standard litres of air per minute are used and an overpressure of 0.2 bar is set. The pH is kept constant at 7.0 with 10% (w/w) phosphoric acid and 25% (w/w) ammonia water. Pluronic® PE 8100 is used as an antifoaming agent. Cultivation is carried out at 37° C. At an optical density of OD600 of 9.0-10.0, 0.1 mM IPTG is induced. Cultivation is terminated after stationary phase is reached.

1. A method for preparing a recombinant hydroxylated collagen peptide component in prokaryotic systems comprising the steps of:
(a) providing a prokaryotic expression system comprising at least one nucleotide sequence encoding at least one recombinant collagen peptide component and at least one nucleotide sequence encoding at least one prolyl 4-hydroxylase, wherein the nucleotide sequence encoding at least one recombinant collagen peptide component comprises the nucleotide sequence of at least one collagen peptide,
b) cultivating the prokaryotic expression system in a culture medium under conditions which allow expression of the at least one recombinant collagen peptide component and the at least one prolyl 4-hydroxylase to obtain at least one hydroxylated collagen peptide component, wherein the collagen peptide component has a reduced hydrophobicity and/or a reduced proline content relative to the at least one collagen peptide encoded by the collagen peptide component encoding nucleotide sequence,
c) recovering the hydroxylated collagen peptide component,
wherein the at least one collagen peptide has the amino acid sequence motif (Gly-X-Y)n and at least 55% of the hydroxylations in the at least one collagen peptide are at a proline in the Y-position.

2. The method according to claim 1, wherein in the nucleotide sequence encoding the collagen peptide component of the prokaryotic expression system, the nucleotide sequence encoding a collagen peptide is fused to at least one nucleotide sequence encoding a peptide residue.

3. The method of claim 2, wherein the peptide residue has at least one of a protein tag, a signal peptide, and/or a lamination domain.

4. The method according to claim 2, wherein the peptide residue is cleavable, in particular enzymatically cleavable.

5. The method according to claim 2, wherein after step c) or after step b) and before step c), a cleavage of the at least one peptide residue of the hydroxylated collagen peptide component takes place.

6. The method of claim 2, wherein the hydroxylated collagen peptide component comprises a collagen peptide and at least one N- and/or C-terminal secretion signal peptide.

7. The method according to claim 1, wherein the collagen peptide encoded by the nucleotide sequence has an amino acid sequence occurring in bovine collagen, in particular in bovine type I collagen, preferably in the α1-chain of bovine type I collagen.

8. The method according to claim 1, wherein at least 70% of the hydroxylations in the at least one collagen peptide are at a proline in the Y-position.

9. The method according to claim 1, wherein the nucleotide sequence encoding prolyl-4-hydroxylase is a nucleotide sequence from Arabidopsis thaliana.

10. The method according to claim 1, wherein the prolyl 4-hydroxylase has an amino acid sequence according to SEQ ID No. 15.

11. A method for preparing a recombinant hydroxylated collagen peptide component in prokaryotic systems comprising the steps of:
i) providing a prokaryotic expression system comprising at least one nucleotide sequence encoding at least one recombinant collagen peptide component and at least one nucleotide sequence encoding at least one proline 4-hydroxylase (PIN4H), wherein the nucleotide sequence encoding at least one recombinant collagen peptide component comprises the nucleotide sequence of at least one collagen peptide,
ii) cultivating the prokaryotic expression system in a culture medium under conditions which allow expression of the at least one recombinant collagen peptide component and the at least one proline 4-hydroxylase (PIN4H) to obtain at least one hydroxylated collagen peptide component, wherein the collagen peptide component has a reduced hydrophobicity and/or a reduced proline content relative to the at least one collagen peptide encoded by the collagen peptide component encoding nucleotide sequence,
iii) recovering the hydroxylated collagen peptide component.

12. The method of claim 11, wherein the at least one proline 4-hydroxylase is a monomeric proline 4-hydroxylase.

13. The method of claim 11, wherein the at least one proline 4-hydroxylase is of bacterial origin.

14. The method of claim 13, wherein the at least one proline 4-hydroxylase is a proline 4-hydroxylase from Streptomyces griseoviridis, Dactylosporangium sp., Pseudomonas stutzeri, Bordetella bronchiseptica, Bradyrhizobium japonicum, Aeromonas caviae, Janthinobacterium sp. or Achromobacter xylosoxidans.

15. The method of claim 11, wherein the at least one proline 4-hydroxylase has an amino acid sequence selected from SEQ ID Nos. 10 to 13, preferably consisting of an amino acid sequence selected from SEQ ID Nos. 10 to 13.

16. A hydroxylated collagen peptide component prepared by a method according to claim 1.

17. A hydroxylated collagen peptide component prepared by the method according to claim 11.


SEQUENCE LISTING (33 sequences)

SEQ ID NO: 1 - protein, 60 aa, Escherichia coli
LAYGSQDNLNPLINEISKIISAAGNFDVKEERAAASLLQLSGNASDFSYGRNSITLTASA

SEQ ID NO: 2 - protein, 218 aa, Escherichia coli
GNSLAKNVLSGGKGNDKLYGSEGADLLDGGEGNDLLKGGYGNDIYRYLSGYGHHIIDDDGGKDDKLSLADIDFRDVAFRREGNDLIMYKAEGNVLSIGHKNGITFRNWFEKESGDISNHQIEQIFDKDGRVITPDSLKKALEYQQSNNKASYVYGNDALAYGSQDNLNPLINEISKIISAAGNFDVKEERAAASLLQLSGNASDFSYGRNSITLTASA

SEQ ID NO: 3 - protein, 707 aa, Escherichia coli
MDSCHKIDYGLYALEILAQYHNVSVNPEEIKHRFDTDGTGLGLTSWLLAAKSLELKVKQVKKTIDRLNFISLPALVWREDGRHFILTKVSKEANRYLIFDLEQRNPRVLEQSEFEALYQGHIILIASRSSVTGKLAKFDFTWFIPAIIKYRKIFIETLVVSVFLQLFALITPLFFQVVMDKVLVHRGFSTLNVITVALSVVVVFEIILSGLRTYIFAHSTSRIDVELGAKLFRHLLALPISYFESRRVGDTVARVRELDQIRNFLTGQALTSVLDLLFSFIFFAVMWYYSPKLTLVILFSLPCYAAWSVFISPILRRRLDDKFSRNADNQSFLVESVTAINTIKAMAVSPQMTNIWDKQLAGYVAAGFKVTVLATIGQQGIQLIQKTVMIINLWLGAHLVISGDLSIGQLIAFNMLAGQIVAPVIRLAQIWQDFQQVGISVTRLGDVLNSPTESYHGKLALPEINGNITFRNIRFRYKPDSPVILDNINLSIKQGEVIGIVGRSGSGKSTLTKLIQRFYIPENGQVLIDGHDLALADPNWLRRQVGVVLQDNVLLNRSIIDNISLANPGMSVEKVIYAAKLAGAHDFISELREGYNTIVGEQGAGLSGGQRQRIAIARALVNNPKILIFDEATSALDYESEHIIMRNMHKICKGRTVIIIAHRLSTVKNADRIIVMEKGKIVEQGKHKELLSEPESLYSYLYQLQSD

SEQ ID NO: 4 - protein, 478 aa, Escherichia coli
MKTWLMGFSEFLLRYKLVWSETWKIRKQLDTPVREKDENEFLPAHLELIETPVSRRPRLVAYFIMGFLVIAVILSVLGQVEIVATANGKLTLSGRSKEIKPIENSIVKEIIVKEGESVRKGDVLLKLTALGAEADTLKTQSSLLQTRLEQTRYQILSRSIELNKLPELKLPDEPYFQNVSEEEVLRLTSLIKEQFSTWQNQKYQKELNLDKKRAERLTILARINRYENLSRVEKSRLDDFRSLLHKQAIAKHAVLEQENKYVEAANELRVYKSQLEQIESEILSAKEEYQLVTQLFKNEILDKLRQTTDNIELLTLELEKNEERQQASVIRAPVSGKVQQLKVHTEGGVVTTAETLMVIVPEDDTLEVTALVQNKDIGFINVGQNAIIKVEAFPYTRYGYLVGKVKNINLDAIEDQKLGLVFNVIVSVEENDLSTGNKHIPLSSGMAVTAEIKTGMRSVISYLLSPLEESVTESLHER

SEQ ID NO: 5 - protein, 238 aa, Artificial Sequence
  Synthetic
MRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 6 - protein, 375 aa, Bacillus subtilis
EGNTREDNFKHLLGNDNVKRPSEAGALQLQEVDGQMTLVDQHGEKIQLRGMSTHGLQWFPEILNDNAYKALANDWESNMIRLAMYVGENGYASNPELIKSRVIKGIDLAIENDMYVIVDWHVHAPGDPRDPVYAGAEDFFRDIAALYPNNPHIIYELANEPSSNNNGGAGIPNNEEGWNAVKEYADPIVEMLRDSGNADDNIIIVGSPNWSQRPDLAADNPIDDHHTMYTVHFYTGSHAASTESYPPETPNSERGNVMSNTRYALENGVAVFATEWGTSQANGDGGPYFDEADVWIEFLNENNISWANWSLTNKNEVSGAFTPFELGKSNATSLDPGPDQVWVPEELSLSGEYVRARIKGVNYEPIDRTKYTKVL

SEQ ID NO: 7 - protein, 367 aa, Escherichia coli
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQT

SEQ ID NO: 8 - protein, 260 aa, Artificial Sequence
  Synthetic
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTGGRFYYAKVASVTDAGVQPVYSLRVDTADHAFITNGFVSHATGLTGLNSGLTTNPGVSAWQVNTAYTAGQLVTYNGKTYKCLQPHTSLAGWEPSNVPALWQLQ

SEQ ID NO: 9 - protein, 505 aa, Artificial Sequence
  Synthetic
CITGDALVALPEGESVRIADIVPGARPNSDNAIDLKVLDRHGNPVLADRLFHSGEHPVYTVRTVEGLRVTGTANHPLLCLVDVAGVPTLLWKLIDEIKPGDYAVIQRSAFSVDCAGFARGKPEFAPTTYTVGVPGLVRFLEAHHRDPDAQAIADELTDGRFYYAKVASVTDAGVQPVYSLRVDTADHAFITNGFVSHATGLTGLNSGLTTNPGVSAWQVNTAYTAGQLVTYNGKTYKCLQPHTSLAGWEPSNVPALWQLQIEGRISHMRKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 10 - protein, 268 aa, Streptomyces griseoviridis
MSVSAPLLDAKVRYGRDGWLPLPHTLSDPDVRKLRQRIEGISREQRPEVVLEEGSSAVRALHGCHDFDEVCARLVRLPALVGLAEQLLGGPVYVYQFKVNMKQAHEGAAWPWHQDFAFWHHEDGMGAPDAVNIAIFLDDVTDENGPLEVIPGSQHAGIVEDTARPGRERSHDWRHHVSAKLEYVVPDEIAGRLAGTFGVRRLTGPAGTAVAFHPSIIHSSSNNTSAQRRCVLLITYNRVTNTPAHPVRPPFLVSRDSTPVVPVDADRL

SEQ ID NO: 11 - protein, 272 aa, Dactylosporangium salmoneum
MLTPTELKQYREAGYLLIEDGLGPREVDCLRRAAAALYAQDSPDRTLEKDGRTVRAVHGCHRRDPVCRDLVRHPRLLGPAMQILSGDVYVHQFKINAKAPMTGDVWPWHQDYIFWAREDGMDRPHVVNVAVLLDEATHLNGPLLFVPGTHELGLIDVERRAPAGDGDAQWLPQLSADLDYAIDADLLARLTAGRGIESATGPAGSILLFDSRIVHGSGTNMSPHPRGVVLVTYNRTDNALPAQAAPRPEFLAARDATPLVPLPAGFALAQPV

SEQ ID NO: 12 - protein, 305 aa, Pseudomonas stutzeri
MNPMQADLYPSRQEDQPSWQERLDPVVYRSDLENAPIAAELVERFERDGYLVIPNLFSADEVALFRAELERMRQDPAVAGSGKTIKEPDSGAIRSVFAIHKDNELFARVAADERTAGIARFILGGDLYVHQSRMNFKPGFTGKEFYWHSDFETWHIEDGMPRMRCLSCSILLTDNEPHNGPLMLMPGSHKHYVRCVGATPENHYEKSLRKQEIGIPDQNSLSELASRFGIDCATGPAGSVVFFDCNTMHGSNGNITPSARSNLFYVYNHVDNAVQAPFCEQKPRPAFVAERENFKPLDIRPQQYL

SEQ ID NO: 13 - protein, 308 aa, Bordetella bronchiseptica
MISPAQDPYASRTDRSSAIIARQDPVVYGEGKFADALSADQVQSYERDGFLLLENLFSDEEVAALLAEVERMTRDPAIVRREEAITEPGSNAVRSIFMVHVLSPILGRLVRDPRLANAARQILGSEVYIHQSRANMKPGFKGKEFYWHSDFETWHVEDGMPSMRALSCSVLLTDNNETNGPLMLVPGSHRQFISCVGETPRDHYKQSLKKQEYGVPDPVSLQLLAEQGGISTMTAKAGSVVFFDCNTMHGSNSNISPWPRANVFMVYNSMENTLNPPKYGLNPRPEHIATRQAFKAVRPLDSLKLVER

SEQ ID NO: 14 - DNA, 849 bp, Arabidopsis thaliana
gcgccggctatgaaaatcgtctttggtttattaacgtttgtcaccgtcggtatggtcatcggttctttattgcaattagcttttatcaaccgcttagaagattcctacggtaccggttttccaagcctccgcggtctgcggggtcaaaatactcgatatctccgggatgtctctcggtgggctaacgacaaagatgctgaattattgcggatcggaaacgtcaaaccggaagtagtcagctggtctccgcggatcatcgttctgcatgactttttaagcccagaagaatgcgaatacctcaaagctatcgctcggccgcgactgcaagtgtctaccgtggtggatgtaaaaactggtaaaggtgtgaaaagcgacgttcgcacgtcttctggtatgtttctcacccatgtagaacgcagctatccgataatacaagctatcgaaaaacggatcgctgtctttagccaagtaccggctgaaaacggtgaactgatccaagtgctccgctatgaaccacaacaattttacaaaccgcatcacgattactttgcggacacctttaacctcaaacgcggtggtcaacgagtagctaccatgctgatgtatctcaccgatgacgtcgaaggtggtgaaacctattttccgttagcgggtgatggtgattgcacttgcggtggaaaaatcatgaaaggaatcagcgtgaaacccactaaaggtgacgctgtgttattttggtctatgggtttagacggtcaaagcgatccgcgctctatacatggtggttgcgaagtgctcagcggtgaaaaatggagcgcgaccaaatggatgcgccaaaaagcgaccagctga

SEQ ID NO: 15 - protein, 282 aa, Arabidopsis thaliana
APAMKIVFGLLTFVTVGMVIGSLLQLAFINRLEDSYGTGFPSLRGLRGQNTRYLRDVSRWANDKDAELLRIGNVKPEVVSWSPRIIVLHDFLSPEECEYLKAIARPRLQVSTVVDVKTGKGVKSDVRTSSGMFLTHVERSYPIIQAIEKRIAVFSQVPAENGELIQVLRYEPQQFYKPHHDYFADTFNLKRGGQRVATMLMYLTDDVEGGETYFPLAGDGDCTCGGKIMKGISVKPTKGDAVLFWSMGLDGQSDPRSIHGGCEVLSGEKWSATKWMRQKATS

SEQ ID NO: 16 - protein, 703 aa, Artificial Sequence
  Synthetic
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGHHHHHHENLYFQSAPAMKIVFGLLTFVTVGMVIGSLLQLAFINRLEDSYGTGFPSLRGLRGQNTRYLRDVSRWANDKDAELLRIGNVKPEVVSWSPRIIVLHDFLSPEECEYLKAIARPRLQVSTVVDVKTGKGVKSDVRTSSGMFLTHVERSYPIIQAIEKRIAVFSQVPAENGELIQVLRYEPQQFYKPHHDYFADTFNLKRGGQRVATMLMYLTDDVEGGETYFPLAGDGDCTCGGKIMKGISVKPTKGDAVLFWSMGLDGQSDPRSIHGGCEVLSGEKWSATKWMRQKATS

SEQ ID NO: 17 - protein, 86 aa, Bos taurus
QEEGQEEGQEEDIPPVTCVQNGLRYHDRDVWKPVPCQICVCDNGNVLCDDVICDELKDCPNAKVPTDECCPVCPEGQESPTDQETT

SEQ ID NO: 18 - protein, 74 aa, Streptococcus pyogenes
ADEQEEKAKVRTELIQELAQGLGGIEKKNFPTLGDEDLDHTYMTKLLTYLQEREQAENSWRKRLLKGIQDHALD

SEQ ID NO: 19 - DNA, 1998 bp, Artificial Sequence
  Synthetic
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggcgaaccgggttcaccgggtgagaatggcgctcctggccagatgggaccgcgtggcttaccaggtgaacgcggtcgtcccggtgctccgggtcctgccggtgcaagaatcgagggaaggatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataa

SEQ ID NO: 20 - DNA, 2787 bp, Artificial Sequence
  Synthetic
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggggcgaaccgggttcaccgggtgagaatggcgctcctggccagatgggaccgcgtggcttaccaggtgaacgcggtcgtcccggtgctccgggtcctgccggtgcaagatgcatcacgggagatgcactagttgccctacccgagggcgagtcggtacgcatcgccgacatcgtgccgggtgcgcggcccaacagtgacaacgccatcgacctgaaagtccttgaccggcatggcaatcccgtgctcgccgaccggctgttccactccggcgagcatccggtgtacacggtgcgtacggtcgaaggtctgcgtgtgacgggcaccgcgaaccacccgttgttgtgtttggtcgacgtcgccggggtgccgaccctgctgtggaagctgatcgacgaaatcaagccgggcgattacgcggtgattcaacgcagcgcattcagcgtcgactgtgcaggttttgcccgcgggaaacccgaatttgcgcccacaacctacacagtcggcgtccctggactggtgcgtttcttggaagcacaccaccgagacccggacgcccaagctatcgccgacgagctgaccggcgggcggttctactacgcgaaagtcgccagtgtcaccgacgccggcgtgcagccggtgtatagccttcgtgtcgacacggcagaccacgcgtttatcacgaacgggttcgtcagccacgctactggcctcaccggtctgaactcaggcctcacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaaatcgagggaaggatttcacatatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataa

SEQ ID NO: 21 - DNA, 1102 bp, Artificial Sequence
  Synthetic
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaagaagccctgaaagacgcgcagactaattcgagctcgaacaacaacaacaataacaataacaacaacctcgggatcgagggaagggatggtcctgcaggtccgaaaggcgaaccgggcagcccgggtgaaaatggcgcccctggtcagatgggtccgcgcggtctgccgggtgaacgtggtcgtccgggtgcacctggacctgctttagcctatggaagtcaggataatcttaatccattaattaatgaaatcagcaaaatcatttcagctgcaggtaattttgatgttaaagaggaaagagctgcagcttctttattgcagttgtccggtaatgccagtgatttttcatatggacggaactcaataactttgacagcatcagcataag

SEQ ID NO: 22 - DNA, 1482 bp, Artificial Sequence
  Synthetic
atggaaggaaacactcgtgaagacaattttaaacatttattaggtaatgacaatgttaaacgcccttctgaggctggcgcattacaattacaagaagtcgatggacaaatgacattagtagatcaacatggagaaaaaattcaattacgtggaatgagtacacacggattacaatggtttcctgagatcttgaatgataacgcatacaaagctcttgctaacgattgggaatcaaatatgattcgtctagctatgtatgtcggtgaaaatggctatgcttcaaatccagagttaattaaaagcagagtcattaaaggaatagatcttgctattgaaaatgacatgtatgtcatcgttgattggcatgtacatgcacctggtgatcctagagatcccgtttacgctggagcagaagatttctttagagatattgcagcattatatcctaacaatccacacattatttatgagttagcgaatgagccaagtagtaacaataatggtggagctgggattccaaataatgaagaaggttggaatgcggtaaaagaatacgctgatccaattgtagaaatgttacgtgatagcgggaacgcagatgacaatattatcattgtgggtagtccaaactggagtcagcgtcctgacttagcagctgataatccaattgatgatcaccatacaatgtatactgttcacttctacactggttcacatgctgcttcaactgaaagctatccgcctgaaactcctaactctgaaagaggaaacgtaatgagtaacactcgttatgcgttagaaaacggagtagcagtatttgcaacagagtggggaactagccaagcaaatggagatggtggtccttactttgatgaagcagatgtatggattgagtttttaaatgaaaacaacattagctgggctaactggtctttaacgaataaaaatgaagtatctggtgcatttacaccattcgagttaggtaagtctaacgcaacaagtcttgacccagggccagaccaagtatgggtaccagaagagttaagtctttctggagaatatgtacgtgctcgtattaaaggtgtgaactatgagccaatcgaccgtacaaaatacacgaaagtacttcaccagcaccagcaccaggagaacctgtacttccaatctgggccacgagggttaccaggtccgccaggtgctccagggccgcaagggtttcaaggtccgccaggggaaccaggtgaaccaggggcgtctgggccgatggggccgcgagggccgccaggtccgccaggtaaaaacggggacgacggtgaggctgggaaaccagggcgaccaggggaacgggggccaccggggccacagggggctcgggggcttccagggacggctggtttaccagggatgaaagggcatcgggggttttctgggcttgacggggcgaaaggggacgctgggccagctggtccgaaaggtgaatga

SEQ ID NO: 23 - DNA, 2112 bp, Artificial Sequence
  Synthetic
atgaaaataaaaacaggtgcacgcatcctcgcattatccgcattaacgacgatgatgttttccgcctcggctctcgccaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggtgaaagatccgcgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggcatcaccatcaccatcacgagaacctgtacttccaatcagcgccggctatgaaaatcgtctttggtttattaacgtttgtcaccgtcggtatggtcatcggttctttattgcaattagcttttatcaaccgcttagaagattcctacggtaccggttttccaagcctccgcggtctgcggggtcaaaatactcgatatctccgggatgtctctcggtgggctaacgacaaagatgctgaattattgcggatcggaaacgtcaaaccggaagtagtcagctggtctccgcggatcatcgttctgcatgactttttaagcccagaagaatgcgaatacctcaaagctatcgctcggccgcgactgcaagtgtctaccgtggtggatgtaaaaactggtaaaggtgtgaaaagcgacgttcgcacgtcttctggtatgtttctcacccatgtagaacgcagctatccgataatacaagctatcgaaaaacggatcgctgtctttagccaagtaccggctgaaaacggtgaactgatccaagtgctccgctatgaaccacaacaattttacaaaccgcatcacgattactttgcggacacctttaacctcaaacgcggtggtcaacgagtagctaccatgctgatgtatctcaccgatgacgtcgaaggtggtgaaacctattttccgttagcgggtgatggtgattgcacttgcggtggaaaaatcatgaaaggaatcagcgtgaaacccactaaaggtgacgctgtgttattttggtctatgggtttagacggtcaaagcgatccgcgctctatacatggtggttgcgaagtgctcagcggtgaaaaatggagcgcgaccaaatggatgcgccaaaaagcgaccagctga

SEQ ID NO: 24 - DNA, 804 bp, Artificial Sequence
  Synthetic
atgtctgtgtccgcgcccctgctcgacgcgaaggtgcgttacggccgggacggatggctgccgttgccccacacgctcagcgatccggacgtcaggaaactccggcagcgcatcgaggggatcagccgggagcagcgccccgaagtggtcctcgaagaagggtcgtcggccgtacgggccttgcacggctgccacgacttcgacgaggtctgcgcccggctcgtccgcctgcccgccctcgtcggcctcgccgaacagctcctcggcggcccggtgtacgtctaccagttcaaggtcaacatgaagcaggcgcacgagggcgccgcctggccgtggcatcaggacttcgcgttctggcaccacgaggacgggatgggcgcccccgacgcggtcaacatcgccatcttcctcgacgacgtcaccgacgagaacggtccgctcgaagtcatacccggcagccagcacgccgggatcgtcgaggacaccgcccggccgggccgcgagcgcagccacgactggcgccaccacgtctcggccaagctggagtacgtcgtaccggacgagatcgccggccggctggcgggcaccttcggggtgcggcgcctcaccggacccgccggcacggccgtggccttccaccccagcatcatccactcctcctccaacaacacctccgcacagcgccgttgcgtgctgctcatcacctacaaccgcgtcaccaacaccccggcgcacccggtcaggccgccgttcctggtcagcagggacagcactccggtggtcccggtggacgccgaccggctc

SEQ ID NO: 25 - protein, 33 aa, Bos taurus
GPRGPPGPPGKNGDDGEAGKPGRPGERGPPGPQ

SEQ ID NO: 26 - protein, 40 aa, Bos taurus
AGPAGPKGEPGSPGENGAPGQMGPRGLPGERGRPGAPGPA

SEQ ID NO: 27 - protein, 36 aa, Bos taurus
GEPGSPGENGAPGQMGPRGLPGERGRPGAPGPAGAR

SEQ ID NO: 28 - protein, 44 aa, Bos taurus
IAGAPGFPGARGPSGPQGPSGPPGPKGNSGEPGAPGSKGDTGAK

SEQ ID NO: 29 - protein, 36 aa, Bos taurus
GLTGSPGSPGPDGKTGPPGPAGQDGRPGPPGPPGAR

SEQ ID NO: 30 - protein, 39 aa, Bos taurus
GERGEQGPAGSPGFQGLPGPAGPPGEAGKPGEQGVPGDL

SEQ ID NO: 31 - protein, 42 aa, Bos taurus
GFPGAAGRVGPPGPSGNAGPPGPPGPAGKEGSKGPRGETGPA

SEQ ID NO: 32 - DNA, 210 bp, Artificial Sequence
  Synthetic
atgggcagcagccatcatcatcatcatcacagcagcggcctggtgccgcgcggcagcattgcaggcgcaccgggttttccaggcgctcgtggtccaagcggtccgcagggtcctagtggtccaccgggaccgaaaggtaatagcggtgaacctggcgctccgggtagcaaaggcgataccggtgcaaaacaccaccaccaccaccactga

SEQ ID NO: 33 - protein, 69 aa, Artificial Sequence
  Synthetic
MGSSHHHHHHSSGLVPRGSIAGAPGFPGARGPSGPQGPSGPPGPKGNSGEPGAPGSKGDTGAKHHHHHH